SEQUENCE LISTING (8 sequences)

SEQ ID NO: 1 - protein, 543 aa, Homo sapiens
  source 1..543: note = Human "SK1" (KCNN1) amino acid sequence
MNSHSYNGSVGRPLGSGPGALGRDPPDPEAGHPPQPPHSPGLQVVVAKSEPARPSPGSPRGQPQDQDDDEDDEEDEAGRQRASGKPSNVGHRLGHRRALFEKRKRLSDYALIFGMFGIVVMVTETELSWGVYTKESLYSFALKCLISLSTAILLGLVVLYHAREIQLFMVDNGADDWRIAMTCERVFLISLELAVCAIHPVPGHYRFTWTARLAFTYAPSVAEADVDVLLSIPMFLRLYLLGRVMLLHSKIFTDASSRSIGALNKITFNTRFVMKTLMTICPGTVLLVFSISSWIIAAWTVRVCERYHDKQEVTSNFLGAMWLISITFLSIGYGDMVPHTYCGKGVCLLTGIMGAGCTALVVAVVARKLELTKAEKHVHNFMMDTQLTKRVKNAAANVLRETWLIYKHTRLVKKPDQARVRKHQRKFLQAIHQAQKLRSVKIEQGKLNDQANTLTDLAKTQTVMYDLVSELHAQHEELEARLATLESRLDALGASLQALPGLIAQAIRPPPPPLPPRPGPGPQDQAARSSPCRWTPVAPSDCG

SEQ ID NO: 2 - DNA, 1632 bp, synthetic construct
  source 1..1632: mol_type = other DNA; note = Human KCNN1 cDNA nucleotide sequence
atgaacagccacagctacaatggcagcgtggggcggccgctgggcagcgggccgggcgccctgggacgagaccctccggaccctgaggccggccaccccccacaacccccgcacagcccgggcctccaggtggtagtggccaagagtgagccagcccggccctcacccggcagcccccgggggcagccccaggaccaggacgatgacgaggatgatgaggaagatgaggccggcaggcagagagcctcggggaaaccctcaaatgtgggccaccgcctgggccaccggcgggcgctcttcgagaagcggaagcgcctcagcgactatgccctcattttcggcatgtttggcatcgtcgtcatggtgacggagaccgagctgtcctggggggtgtacaccaaggagtctctgtactcattcgcactcaaatgcctcatcagcctctccacggccatcctgctgggtctcgttgtcctctaccatgcccgggagatccagctgttcatggtggacaacggggctgatgactggcgcatcgccatgacctgcgagcgcgtgttcctcatctcgctagagctggcagtgtgcgccattcacccggtgcccggccactaccgcttcacgtggacggcgcggctggccttcacgtacgcgccctcggtggccgaggccgacgtggacgtgctgctgtccatccccatgttcctgcgcctctacctgctgggccgggtgatgctactgcacagcaaaatcttcacggacgcctcgagccgcagcatcggggccctcaacaagatcaccttcaacacgcgcttcgtcatgaagacactcatgaccatctgccccggcaccgtgctgctggtcttcagcatctcctcctggatcatcgcagcctggaccgtgcgcgtctgcgagaggtaccacgacaagcaggaagtgaccagcaacttcctgggggccatgtggctgatttccatcaccttcctctccattggctacggcgacatggtgccccacacctactgcgggaagggtgtgtgcctgctcactggcatcatgggagctggctgtaccgcgctcgtggtggctgtggtggctcggaagctggagctcaccaaggctgagaagcacgtgcacaacttcatgatggacactcagctcaccaagcgggtaaaaaacgccgctgctaacgttctcagggagacgtggctcatctacaaacataccaggctggtgaagaagccagaccaagcccgggttcggaaacaccagcgtaagttcctccaagccatccatcaggctcagaagctccggagtgtgaagatcgagcaagggaagctgaacgaccaggctaacacgcttaccgacctagccaagacccagaccgtcatgtacgaccttgtatcggagctgcacgctcagcacgaggagctggaggcccgcctggccaccctggaaagccgcttggatgcgctgggtgcctctctacaggccctgcctggcctcatcgcccaagccatacgcccacccccgcctcccctgcctcccaggcccggccccggcccccaagaccaggcagcccggagctccccctgccggtggacgcccgtggccccctcggactgcgggtga

SEQ ID NO: 3 - protein, 538 aa, Mus musculus
  source 1..538: note = Mouse "SK1" (Kcnn1) amino acid sequence
MSSHSHNGSVGQPLGSGPGFLGWEPVDPEAGRPLQPTQGPGLQMVAKGQPVRLSPGGSRGHPQEQEEEEEEEEEEEDKTGSGKPPTVSHRLGHRRALFEKRKRLSDYALIFGMFGIVVMVTETELSWGVYTKESLCSFALKCLISLSTVILLGLVILYHAREIQLFLVDNGADDWRIAMTWERVSLISLELVVCAIHPVPGHYRFTWTARLAFSLVPSAAEADLDVLLSIPMFLRLYLLARVMLLHSRIFTDASSRSIGALNRVTFNTRFVTKTLMTICPGTVLLVFSVSSWIVAAWTVRVCERYHDKQEVTSNFLGAMWLISITFLSIGYGDMVPHTYCGKGVCLLTGIMGAGCTALVVAVVARKLELTKAEKHVHNFMMDTQLTKRVKNAAANVLRETWLIYKHTRLVKKPDQGRVRKHQRKFLQAIHQAQKLRSVKIEQGKVNDQANTLAELAKAQSIAYEVVSELQAQQEELEARLAALESRLDVLGASLQALPGLIAQAICPLPPPWPGPGHLATATQSPQSHWLPTMGSDCG

SEQ ID NO: 4 - DNA, 1617 bp, synthetic construct
  source 1..1617: mol_type = other DNA; note = Mouse "SK1" (Kcnn1) cDNA nucleotide sequence
atgagtagccacagccacaatggcagcgtggggcagcctctgggcagcgggcctggattcctgggctgggagcctgtggaccctgaggcaggccgccccctgcagcccacccaaggcccaggcctgcagatggtggccaagggtcagcctgtcaggctgtcacccggtggttccaggggccacccccaggagcaggaggaggaagaggaagaggaggaggaggaggaggacaagacaggctcagggaagcccccaacagtcagccaccgcctgggacaccgcagggccctctttgaaaagcgtaaacggctcagtgactatgcgctcatctttggcatgtttgggattgtcgtcatggtgacagaaacagagctgtcctggggtgtatacaccaaggagtcactctgctcttttgctctgaaatgcctcatcagcctgtccactgtcatcttgcttggccttgtcatcctgtaccacgcccgagagatccagctgttcttggtggacaatggtgccgacgactggcgtatcgccatgacgtgggagcgcgtgtccctgatctcgctggagttggtcgtgtgtgccatccacccggtgcccggccactatcgcttcacgtggacggcacgactggccttctctctggtgccgtcggcagccgaggcagacctggatgtgctgctgtccatccccatgttcctgcgcctctacctgctggctcgggtcatgctcctgcacagccgcatcttcaccgatgcatccagccgcagcatcggggcccttaaccgcgtcaccttcaacacacgcttcgtcaccaagacgctcatgaccatctgccccggcactgtgttgttggtcttcagcgtctcctcctggatcgttgctgcgtggacagtgcgcgtgtgtgagaggtaccacgataagcaggaagtgaccagcaacttcctgggagccatgtggctcatctccatcaccttcttgtccattggctatggagacatggtgccgcatacctactgtgggaagggtgtgtgtctgctcactggcatcatgggagcaggctgcactgcactcgtggtggccgtcgtggctcggaagttggaactcaccaaggctgagaaacacgtgcacaacttcatgatggacacacagctcaccaagcgggtcaaaaatgctgctgcaaacgttctcagggagacatggctcatctacaaacacaccaggctggtgaagaagccagaccaaggccgggttcggaaacaccagcgtaagttccttcaggccatccatcaggctcagaagctccgaagtgtgaagattgaacaagggaaggtgaacgatcaggccaacacgctggctgagctggccaaggcacagagcatcgcatatgaggtggtgtcagagctgcaggcccagcaggaggagttggaggcacgcctagccgccttggagagccgactggatgtcctgggtgcctccctgcaggctctaccaggccttatagcccaagccatatgccctctaccaccaccctggcctgggcctggtcacctggccacagccacccagagcccacaaagccactggctgcccaccatgggatcagactgtgggtga

SEQ ID NO: 5 - DNA, 130 bp, synthetic construct
  source 1..130: mol_type = other DNA; note = AAV2 inverted terminal repeat (ITR) DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 119 bp, synthetic construct
  source 1..119: mol_type = other DNA; note = Modified AAV2 ITR DNA
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 7 - DNA, 112 bp, synthetic construct
  source 1..112: mol_type = other DNA; note = Modified AAV2 ITR that produces self-complementary vectors
cactccctctctgcgcgctcgctcgctcactgaggccgggcgaccaaaggtcgcccgacgcccgggctttgcccgggcggcctcagtgagcgagcgagcgcgcagagaggga

SEQ ID NO: 8 - DNA, 584 bp, synthetic construct
  source 1..584: mol_type = other DNA; note = Cytomegalovirus (CMV) enhancer/promoter sequence
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

(19) United States
(12) Patent Application Publication
(10) Pub. No.: US 2026/0284230 A1
(43) Pub. Date: Sep. 24, 2026

(54) METHODS OF MODIFYING NEURONS IN VIVO TO TREAT AND/OR PREVENT AMYOTROPHIC LATERAL SCLEROSIS (ALS)

(71) Applicant: Yale University, New Haven, CT (US)

(72) Inventors: Arthur L. HORWICH, Woodbridge, CT (US); Wayne A. FENTON, West Haven, CT (US); Maria NAGY, New Haven, CT (US)

(73) Assignee: Yale University, New Haven, CT (US)

(21) Appl. No.: 19/474,346

(22) PCT Filed: Apr. 10, 2024
(86) PCT No.: PCT/US2024/023827

§ 371 Date: Oct. 10, 2025

Related U.S. Application Data
(60) Provisional application No. 63/495,163, filed on Apr. 10, 2023.

(51) Int. Cl. A61K 48/00; A61K 38/17; A61P 25/28

(52) U.S. Cl. CPC A61K 48/0058; A61K 38/1709; A61K 48/0033; A61P 25/28

(57) ABSTRACT

Provided herein are compositions and methods of treating and/or preventing amyotrophic lateral sclerosis (ALS) in a subject in need thereof, the methods comprising administering an agent wherein the agent modifies neurons via changes in Kcnn1 protein expression or activity, which in turn modifies neuron firing and/or increases the clearance or protection from toxicity of an ALS-causing protein.

### CROSS REFERENCE TO RELATED APPLICATIONS

This application claims priority to U.S. Provisional Application No. 63/495,163, filed on Apr. 10, 2023, the entire contents of which are incorporated herein by reference.

### REFERENCE TO SEQUENCE LISTING SUBMITTED ELECTRONICALLY

The contents of the computer readable Sequence Listing in XML format (“XML Document”) submitted electronically herewith are incorporated herein by reference in their entirety. A computer readable format copy of the Sequence Listing (filename: 2681_147PC01_SequenceListing_ST26.xml.xml, date produced: Apr. 9, 2024; size: 14,934 bytes) is submitted per 37 C.F.R. §§ 1.831-1.835.

### FIELD

The present disclosure relates to administering agents to treat and/or prevent ALS, wherein the agent increases neuronal levels of Kcnn1, the subunit of the SK1 channel, and extends motor neuron survival either by affecting firing rate and/or proteostasis of motor neurons.

### BACKGROUND

Amyotrophic lateral sclerosis (ALS), also referred to as Lou Gehrig's disease, is a motor neuron disease that is characterized by dysfunction and death of upper and lower motor neurons that innervate striated muscle. The typical presentation of ALS involves a defect of limb movement, including weakness, twitching, or stiffness. A small percentage of ALS subjects present bulbar defects, affecting swallowing and speech, and a fraction of ALS subjects develop difficulties with thinking and behavior, including, for some, frontotemporal dementia. Eventually, affected subjects are unable to walk, use their hands, speak, swallow, or breathe. Notably, eye movement of ALS subjects is relatively spared.

ALS usually presents in human subjects between 40 and 70 years of age and somewhat earlier in inherited cases; however, the age of onset of the majority of cases is 60-80 years of age. Approximately one out of every 2,000 people develop ALS during their lifetime, and ALS is approximately two times more likely in males than females. Subjects typically present with extremity symptoms, including loss of strength and clumsiness. About 30-40% of motor neurons have already been lost by the time of diagnosis, and the progression suggests that motor neuron loss may proceed at the rate of 50% of remaining motor neurons lost every six months. There is damage and loss of both upper and lower motor neurons. The average survival from onset to death is two to four years. (For additional clinical description of onset, diagnosis (e.g., El Escorial criteria), progression, and management of ALS, see Amyotrophic Lateral Sclerosis, Mitsumoto, Przedborski, and Gordon eds, Taylor and Francis, New York, 2006).

Approximately 10% of cases involve one of the more than 20 genes that have been associated with familial ALS, of which four account for the majority of familial cases: C9orf72 hexanucleotide repeat expansion (40%), and various mutations in SOD1 (20%), FUS (1-5%), and TARDBP (1-5%). Mutations in other genes including profilin, optineurin, VAP-B, ubiquilin-2, VCP also occur, but are less common. The remaining 90% of cases are sporadic, possibly involving environmental factors and/or multiple genetic changes (see Taylor, Brown, Cleveland (2016) Nature 539, 197-206). Both familial and sporadic forms involve general motor system collapse and are not readily distinguishable clinically, albeit that some genetic forms exhibit earlier onset. One possible view of ALS is that it comprises dozens of triggers, all of which produce the same progressive collapse of the motor system. Notably, in most cases, motor neurons accrete abnormal inclusions/aggregates in the cell body, including ubiquitinated inclusions in nearly 100% of patients (see ALS text 2006, chapter 3). In familial forms of ALS, mutationally altered versions of such proteins as SOD1, C9ORF72, FUS, TDP43, or others, are found in such inclusions.

Mouse models offer an approach to investigate the pathological mechanisms in ALS and to identify treatment avenues. To date, ALS has been modeled most successfully in mice that are transgenic for a variety of mutant forms of human Cu/Zn superoxide dismutase 1 (SOD1), allowing for the study of progressive motor neuron loss and paralysis. In particular, these strains of mice exhibit full penetrance and relatively uniform time of paralysis, the latter inversely proportional to copy number of the transgene.

At present, there is no cure for ALS, and a variety of treatments have been directed toward improving symptoms and extending life. Riluzole was the first FDA approved drug for the treatment of ALS, and may extend life by a few months. Riluzole may act on one or more ion channel or neurotransmitter receptors and its therapeutic mechanism remains unclear. Nuedexta, a combination of dextromethorphan and quinidine, has proved useful for bulbar ALS patients, improving speech and swallowing (Smith et al, Neurotherapeutics 2017 October; 14 (4): 952-960). A combination of sodium phenylbutyrate and taurursodiol, marketed as Amylyx 0035, aimed at improving ER and mitochondrial metabolism respectively, was FDA approved, but has failed in a late stage trial to affect outcome any differently than placebo. PIKfyve kinase inhibitors, proposed to clear aggregation-prone proteins via directing multi-vesicular bodies and autophagosomes to apparent exocytosis (Hung et al, Cell 2023 Vol 186 (4), 786-802, E28), have entered early phase trials.

Intrathecal antisense oligonucleotide (ASO) therapy has also been initiated for specific single gene-associated forms of ALS: SOD1, C9orf72, and FUS forms of ALS (see Boros et al, Neurotherapeutics. 2022 July; 19 (4): 1145-1158). In the case of SOD1 forms, the ASO known as Tofersen has been tolerated but missed primary outcome for efficacy in a phase 3 trial; however, there were favorable clinical and biomarker trends. A phase 3 randomized placebo-controlled trial is underway for presymptomatic SOD1 mutation carriers. A phase 1 trial of a C9ORF72 ASO was well-tolerated but none of the clinical endpoints were met and a trend to greater clinical decline was observed. A second phase 1 trial for C9ORF72 ALS achieved reduction of polyGP in CSF but no change in clinical outcome had been observed. In the case of FUS, an initial trial on a respirator-dependent (26 yr. old patient with advanced disease) of an escalating intrathecal dose of an ASO showed lowering of both normal and mutant FUS protein and reduction of insoluble FUS aggregates in the spinal cord and motor cortex at postmortem. Additional patients with FUS mutations, both presymptomatic and symptomatic, have been treated with the same ASO under an Expanded Access Program IND. A phase 3 trial of 64 patients with the same ASO is also underway. ASOs targeting ATXN2 have also been tested in a phase 1 trial on ALS patients with intermediate level polyQ expansion of ATXN2 (Boros et al, 2022). (ATXN2 has been linked to aggravated clinical misbehavior of TDP43 and intermediate polyQ expansion in ATXN2 to increased risk of ALS).

Edaravone, an approved free-radical scavenger, may also provide some improvement. Agents in trial have included pridopidine (sigma-1 receptor agonist), ezogabine (retigabine), a KCNQ activator, RMS60, an anti-inflammatory agent, and ibudilast, a cyclic nucleotide phosphodiesterase inhibitor with anti-inflammatory effects.

Mechanical ventilation for respiratory failure can prolong survival but does not affect disease progression.

Thus, there is a need in the art for improved compositions and methods to improve survival and/or prevent ALS.

### SUMMARY

The present disclosure provides compositions and methods for treating and/or preventing ALS. In aspects described herein, provided are compositions comprising a gene transfer construct, wherein the construct comprises: (a) a human KCNN1 cDNA, or variant thereof; (b) an enhancer-promoter combination or a promoter operably linked to the human KCNN1 cDNA, or variant thereof, wherein the enhancer-promoter combination or promoter is capable of controlling expression of the human KCNN1 cDNA; (c) an inverted terminal repeat (ITR) 5′ of the enhancer-promoter combination or promoter operably linked to the human KCNN1 cDNA, or variant thereof; and (d) an inverted terminal repeat (ITR) 3′ of the human KCNN1 cDNA, or variant thereof.

In certain aspects, the gene transfer construct is an adeno-associated virus (AAV). In some aspects, the AAV is selected from an AAV1, AAV2, AAV4, AAV5, AAV6, AAV7, AAV8, or AAV9 capsid serotype, a recombinant AAV (rAAV), or a functional variant of AAV1, AAV2, AAV4, AAV5, AAV6, AAV7, AAV8, or AAV9, including self-complementary versions thereof.

In certain aspects, the human KCNN1 cDNA comprises a polynucleotide sequence encoding the polypeptide with amino acid sequence of SEQ ID NO: 1 or a variant or fragment having at least about 80%, or at least about 90%, or at least about 95%, or at least about 97%, or at least about 99% identity thereto. In certain aspects, the human KCNN1 cDNA comprises a polynucleotide sequence of SEQ ID NO: 2 or a variant or fragment having at least about 80%, or at least about 90%, or at least about 95%, or at least about 97%, or at least about 99% identity thereto. In some aspects, the human KCNN1 cDNA comprises a codon optimized form of a polynucleotide sequence of SEQ ID NO: 2.

In certain aspects, the enhancer-promoter combination or promoter is selected from a cytomegalovirus (CMV) enhancer/promoter, a CMV enhancer fused to the chicken β-actin promoter (CAG), a chicken β-actin promoter (CBA), a simian vacuolating virus 40 (SV40) enhancer/promoter, a polyubiquitin C gene promoter (UBC), an elongation-factor 1α subunit (EF-1α) promoter, or a phosphoglycerate kinase promoter (PGK). In some aspects, the enhancer-promoter combination or promoter comprises a polynucleotide sequence of SEQ ID NO: 8 or a functional variant having at least about 80%, or at least about 90%, or at least about 95%, or at least about 97%, or at least about 99% identity thereto.

In certain aspects, the enhancer-promoter combination or promoter is suitable for use in humans. In certain aspects, the enhancer-promoter combination or promoter is tissue-specific, inducible, or both tissue-specific and inducible. In some aspects, the tissue-specific promoter or enhancer/promoter is a neuron-specific enhancer-promoter combination or promoter. In some aspects, the neuron-specific enhancer-promoter combination or promoter is selected from a neuron-specific enolase (ENO2), a platelet-derived growth factor α-chain (PDGFA), a platelet-derived growth factor β-chain (PDGFB), a synapsin (SYN1), a methyl-CpG binding protein 2 (MECP2), a Ca2+/calmodulin-dependent protein kinase II (CAMK2G), metabotropic glutamate receptor 2 (GRM2), a neurofilament light (NEFL) or heavy (NEFH) chain, a proenkephalin (PENK), or an excitatory amino acid transporter 2 (SLC1A2).

In certain aspects, one or more of the ITRs comprises an ITR of the same serotype as the AAV capsid, comprises an ITR derived from the same serotype as the AAV capsid, comprises an ITR of a different serotype as the AAV capsid, or comprises an ITR derived from a different serotype as the AAV capsid. In some aspects, one or more of the ITRs comprises a polynucleotide sequence of SEQ ID NO: 5, SEQ ID NO: 6, or SEQ ID NO: 7, or a functional variant having at least about 80%, or at least about 90%, or at least about 95%, or at least about 97%, or at least about 99% identity thereto. In some embodiments, the recombinant viral genome is self-complementary.

In certain aspects, the gene transfer construct further comprises one or more of an intron, a transcriptional termination signal, a polyadenylation (polyA) site, an miRNA, or a post-transcriptional regulatory element (PRE). In some aspects, the gene construct comprises, from 5′ to 3′: an ITR, a CMV enhancer/promoter, an intron, a human KCNN1 cDNA or fragment or variant thereof, a transcriptional termination sequence, a poly A site, and an ITR.

In certain aspects, the compositions are suitable for intrathecal or intracerebroventricular delivery.

The present disclosure also provides pharmaceutical compositions comprising the compositions described herein and a pharmaceutically accepted excipient, carrier or diluent.

The present disclosure also provides an ALS therapy comprising an effective amount of the pharmaceutical compositions described herein.

In addition, the present disclosure provides methods for treating or preventing ALS in a subject in need thereof, comprising administering to the subject an effective amount of the pharmaceutical compositions described herein or the ALS therapy described herein.

The present disclosure also provides methods for treating or preventing ALS in a subject in need thereof, comprising administering to the subject an effective amount of an ALS therapy, wherein the ALS therapy modulates (e.g. increases) the expression of KCNN1 or a fragment or variant thereof.

The present disclosure also provides methods of treating or preventing ALS in a subject in need thereof comprising administering to the subject an effective amount of an ALS therapy, wherein the ALS therapy: decreases and/or inhibits a neuron firing rate; increases and/or stimulates a duration of afterhyperpolarization in a neuron; decreases and/or inhibits membrane potential during the afterhyperpolarization phase in a neuron; decreases and/or inhibits negative membrane potential further below resting potential during an afterhyperpolarization phase; increases and/or prolongs an afterhyperpolarization phase; increases and/or stimulates K+ efflux during afterhyperpolarization phase in a neuron; increases and/or stimulates expression of KCNN1 gene or protein in a neuron; increases and/or stimulates activity of small conductance calcium-gated K+ (SK) channels in a neuron; increases and/or stimulates protection of motor neurons; increases and/or stimulates clearance of pathogenic protein; or a combination thereof. In some aspects, the small conductance calcium-gated K+ (SK) channel is SK1 or a fragment or variant thereof.

In certain aspects, the ALS therapy is: a gene therapy; a triazolo pyrimidine; or one or more of N-{7-[1-(4-chloro-2-methylphenoxy)ethyl]-[1,2,4]triazolo[1,5-a]pyrimidin-2-yl}-N′-methoxy-formamidine [(-)CM-TPMF], chlorzoxazone, 5,6-Dichloro-1-ethyl-1,3-dihydro-2H-benzimidazol-2-one (DCEBIO), riluzole, and/or 1-ethyl-2benzimidazolinone (1-EBIO), or a pharmaceutically acceptable salt thereof. In some aspects, the gene therapy is a viral gene therapy. In some aspects, the viral gene therapy comprises a recombinant adeno-associated virus (rAAV). In some aspects, the rAAV is a human KCNN1-expressing rAAV or an rAAV expressing a fragment or variant of KCNN1. In some aspects, the viral gene therapy comprises a composition as described herein. In some aspects, the triazolo pyrimidine is selected from (-)CM-TPMF or a derivative of (-)CM-TPMF.

In certain aspects of the methods described herein, the methods further comprise administering an effective amount of at least one additional agent or selecting a subject for administration of the ALS therapy, wherein the subject is undergoing treatment with at least one additional agent. In some aspects, the additional agent is a benzothiazole (e.g. riluzole, or a derivative thereof); antioxidant (e.g. edaravone, or a derivative thereof); myeloperoxidase (MPO) enzyme inhibitor (e.g. verdiperstat, or a derivative thereof); catalytically-active gold nanocrystal (e.g. CNMAU-8, or a derivative thereof); sigma-1 receptor agonist (e.g. pridopidine, or a derivative thereof); KCNQ activator (e.g. ezogabine, or a derivative thereof); PIKfyve kinase inhibitor (e.g. YM201636, or a derivative thereof); endosomal trafficking modulator; electrokinetically altered aqueous fluid (e.g. RMS60, or a variant thereof); anti-inflammatory agent; cyclic nucleotide phosphodiesterase inhibitor (e.g. ibudilast, or a derivative thereof); antisense oligonucleotide (e.g. directed to FUS, C9ORF72, or directed to SOD1); antibody (e.g. anti-CD40L, e.g. tegoprubart); trehalose; or combination thereof. In some aspects, the additional agent is selected from N-{7-[1-(4-chloro-2-methylphenoxy)ethyl]-[1,2,4]triazolo[1,5-a]pyrimidin-2-yl}-N′-methoxy-formamidine [(-)CM-TPMF], chlorzoxazone, 5,6-Dichloro-1-ethyl-1,3-dihydro-2H-benzimidazol-2-one (DCEBIO), riluzole, and/or 1-ethyl-2benzimidazolinone (1-EBIO), or a pharmaceutically acceptable salt thereof.

In certain aspects of the methods described herein, the ALS therapy, the additional agent, or the ALS therapy and the additional agent is administered to the subject by at least one route selected from nasal, inhalational, topical, oral, buccal, rectal, pleural, peritoneal, vaginal, intramuscular, subcutaneous, transdermal, epidural, intratracheal, optic, intraocular, intracranial, intrathecal, intracerebroventricular, or intravenous.

In certain aspects of the methods described herein, the subject is a mammal. In some aspects, the mammal is a human. In some aspects, the human is about 40 to about 80 years old.

In certain aspects of the methods described herein, the ALS is sporadic ALS (sALS) or familial ALS (fALS). In other aspects of the methods described herein, the ALS is early, middle, or late stage. In some aspects of the methods described herein, the method prevents or retards progression from early to middle stage ALS or middle to late-stage ALS. In some aspects, the subject demonstrates one or more of loss of strength; clumsiness; difficulty breathing; neurogenic atrophy; or loss of motor neurons in primary motor cortex, the brainstem, and/or the spinal cord. In some aspects, the subject presents one or more of the following genetic risk factors: C9orf72 hexanucleotide repeat expansion; one or more mutations in SOD1; one or more mutations in FUS; one or more mutations in TARDBP; or one or more mutations in other genes associated with or conferring risk for ALS.

In certain aspects of the methods described herein, the subject has failed and/or responded poorly to a previous therapy. In some aspects, the subject has failed and/or responded poorly to a benzothiazole (e.g. riluzole) or an antioxidant (e.g. edaravone) or a combination therapy of phenylbutyrate/taurursodiol, or an intrathecal ASO.

In certain aspects of the methods described herein, the subject demonstrates one or more of the following during or after treatment: decreased neuron firing rate; increased duration of afterhyperpolarization in a neuron; decreased membrane potential during afterhyperpolarization phase in a neuron; decreased negative membrane potential further below resting potential during the AHP phase; increased and/or prolonged afterhyperpolarization phase; increased and/or stimulated K+ efflux during afterhyperpolarization phase in a neuron; increased and/or stimulated expression of KCNN1 in a neuron; increased and/or stimulated activity of small conductance calcium-gated K+ (SK) channels in a neuron; increased and/or stimulated protection of motor neurons; or increased and/or stimulated clearance of pathogenic protein.

In certain aspects of the methods described herein, the neuron is a cortical or spinal cord motor neuron, or a brainstem motor neuron.

In certain aspects of the methods described herein, the method further comprises evaluating analysis of a biological fluid, e.g., without limitation, for biomarkers, e.g., one or more of NfL and GPNMB. In some aspects, the biological fluid is cerebrospinal fluid (CSF) or blood.

In certain aspects of the methods described herein, the method further comprises evaluating analysis of a brain image. In some aspects, the brain image is from one or more of computed tomography (CT), positron emission tomography (PET), or magnetic resonance imaging (MRI).

The present disclosure also provides methods of making an ALS therapy, comprising: (a) identifying the ALS therapy by: (i) administering an effective amount of a test agent to a transgenic animal expressing G93A mutant human SOD1 or a wild-type animal; (ii) identifying the test agent as an ALS therapy if the transgenic animal demonstrates one or more of increased expression of Kcnn1, increased amount or activity of the gene product (KCNN1 protein) or of a fragment or variant thereof, improved survival time to paralysis, as compared to a control; and (b) formulating the candidate agent for administration for the treatment of ALS.

In embodiments, e.g. when overexpression of Kcnn1 confers protection via an increased amount of channel activity, there is provided a method of identifying an ALS therapy comprising: (a) identifying a candidate agent that modulates Kcnn1 channel activity comprising: (i) providing a stably transfected Kcnn1-expressing cell line or cells transiently expressing Kcnn1, (ii) treating the cells with one or more test agents, (iii) measuring a potassium export current activity (e.g. under voltage clamp conditions) that can be shown at a next level to be calcium dependent, as compared to absence of the test agent and/or compared to the positive control SK1 activator agent CM-TPMF (e.g. Hougaard et al, 2012 Mol. Pharm. 81, 210-219) (b) administering an effective amount of the candidate agent to a transgenic animal expressing G93A mutant human SOD1 or administering vehicle; (c) classifying the candidate agent as an ALS therapy if the transgenic animal demonstrates one or more of increased expression of Kcnn1, increased channel activity of Kcnn1 in spinal cord tissue slices from mice exposed to compound or vehicle (see Hadzipasic et al, PNAS, 2014 111 (47) 16883-16888), and/or improved survival time (time to paralysis), as compared to the nontreated G93A mouse; and (d) formulating the ALS therapy for administration for the treatment of ALS.

In embodiments, e.g. when overexpression of Kcnn1 confers neuroprotection via an effect of overexpression of Kcnn1 membrane protein subunit, folded and assembled or not, localized to membranes or not, effecting activation of a stress response that is protective, e.g. by induction of proteostatic pathways, which might include diminished translation or increased clearance of the proteotoxic G85R SOD1YFP or G93A SOD1, there is provided a method of identifying an ALS therapy comprising: (i) transducing cells with G85R SOD1YFP; (ii) treating the cells with chemical compounds that abolish G85R SOD1YFP fluorescence, in the same manner that cotransduction with Kcnn1 effects such abolition; and inspecting the cells for the same morphologic or informatic changes that are effected by Kcnn1 (see, e.g., FIGS. 18A-D for morphology changes).

The present disclosure also provides methods of identifying an ALS therapy that is effective in treating or preventing ALS comprising administering an effective amount of the agent to an SOD1-mutant mouse and determining time to paralysis, wherein an increased time to paralysis compared to an SOD1-mutant mouse that does not receive the agent indicates that the agent is an ALS therapy.

In certain aspects of the methods described herein, the ALS therapy is a gene therapy, a biologic agent, a small molecule, or a polynucleotide agent. In some aspects, the gene therapy is a viral gene therapy. In some aspects, the viral gene therapy is or comprises a recombinant adeno-associated virus (rAAV). In some aspects, the rAAV is a Kcnn1-expressing rAAV. In some aspects, the biologic agent is an antibody or peptide. In some aspects, the polynucleotide agent is selected from mRNA, siRNA, shRNA, miRNA, or cDNA.

In aspects, the present disclosure provides a pharmaceutical composition comprising a gene transfer construct, wherein the gene transfer construct comprises: (a) a human KCNN1 cDNA, or functional variant thereof having a polynucleotide sequence encoding the polypeptide of SEQ ID NO: 1 or a functional variant having at least about 95%, identity thereto; (b) an enhancer-promoter combination or a promoter operably linked to the human KCNN1 cDNA, or functional variant thereof, wherein the enhancer-promoter combination or promoter is capable of controlling expression of the human KCNN1 cDNA; (c) an inverted terminal repeat (ITR) 5′ of the enhancer-promoter combination or promoter operably linked to the human KCNN1 cDNA, or functional variant thereof; and (d) an inverted terminal repeat (ITR) 3′ of the human KCNN1 cDNA, or functional variant thereof; wherein the gene transfer construct is an adeno-associated virus 9 (AAV9). In some embodiments, the human KCNN1 cDNA comprises a polynucleotide sequence encoding the polypeptide of SEQ ID NO: 1. In some embodiments, the human KCNN1 cDNA comprises a polynucleotide sequence of SEQ ID NO: 2 or a functional variant or fragment having at least about 95% identity thereto. In some embodiments, the enhancer-promoter combination or promoter comprises a polynucleotide sequence of SEQ ID NO: 8 or a functional variant or fragment having at least about 95% identity thereto. In some embodiments, the one or more of the ITRs comprises a polynucleotide sequence of SEQ ID NO: 5, SEQ ID NO: 6, or SEQ ID NO: 7 or a functional variant or fragment having at least about 95% identity thereto.

In aspects, the present disclosure provides a method for treating or preventing ALS in a subject in need thereof, comprising administering to the subject an effective amount of a pharmaceutical composition comprising a gene transfer construct, wherein the gene transfer construct comprises: (a) a human KCNN1 cDNA, or functional variant thereof having a polynucleotide sequence encoding the polypeptide of SEQ ID NO: 1 or a functional variant having at least about 95%, identity thereto; (b) an enhancer-promoter combination or a promoter operably linked to the human KCNN1 cDNA, or functional variant thereof, wherein the enhancer-promoter combination or promoter is capable of controlling expression of the human KCNN1 cDNA; (c) an inverted terminal repeat (ITR) 5′ of the enhancer-promoter combination or promoter operably linked to the human KCNN1 cDNA, or functional variant thereof; and (d) an inverted terminal repeat (ITR) 3′ of the human KCNN1 cDNA, or functional variant thereof; wherein the gene transfer construct is an adeno-associated virus 9 (AAV9). In some embodiments, the human KCNN1 cDNA comprises a polynucleotide sequence encoding the polypeptide of SEQ ID NO: 1. In some embodiments, the human KCNN1 cDNA comprises a polynucleotide sequence of SEQ ID NO: 2 or a functional variant or fragment having at least about 95% identity thereto. In some embodiments, the enhancer-promoter combination or promoter comprises a polynucleotide sequence of SEQ ID NO: 8 or a functional variant or fragment having at least about 95% identity thereto. In some embodiments, the one or more of the ITRs comprises a polynucleotide sequence of SEQ ID NO: 5, SEQ ID NO: 6, or SEQ ID NO: 7 or a functional variant or fragment having at least about 95% identity thereto.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates fluorescent micrographs of oculomotor (3N), facial (7N), and hypoglossal (12N) motor neurons in mice with G85R SOD1YFP-linked ALS. Aggregation of G85R SOD1YFP protein was observed by yellow fluorescent protein (YFP) fluorescence (upper micrographs) in 7N and 12N motor neurons but not in 3N motor neurons in 3-month-old G85R SOD1YFP (B6SJL) transgenic mice. The lower micrographs show anti-choline acetyltransferase (ChAT) immunostaining of the same sections to identify large (cholinergic) motor neurons. FIG. 1B displays a graph showing the percentage of motor neurons containing aggregates in the spared (3N, 4N, 6N) versus vulnerable (5N, 7N, 12N) motor neuron nuclei in three G85R SOD1YFP mice. For each condition (i.e. 3N, 4N, 6N, 5N, 7N, and 12N) three mice are shown from left to right: Mouse 1, Mouse 2, and Mouse 3.

FIG. 2 illustrates Kaplan-Meier survival plots for control G85R SOD1YFP homozygous mice and G85R SOD1YFP mice homozygous for candidate knockout genes Chrm2 KO, Kcnb1 KO, Kcng1 KO, Dkk3 KO, Gabra2 KO, and Stk32a KO. In normal mice, these six genes exhibited relatively less RNA expression in 3N (spared in ALS) versus 12N and spinal cord motor neurons (vulnerable in ALS). Median survival to the time of paralysis (days) of the knock-out mouse strain was compared to that of the control G85R SOD1YFP homozygous mice, and p-values for the plots of the knock-out strain relative to the plot of the control G85R SOD1YFP homozygous line are presented for each candidate gene.

FIG. 3 illustrates Kaplan-Meier survival plots for control G85R SOD1YFP homozygous mice and G85R SOD1YFP mice hemizygous for transgenic candidate genes (Thy 1.2 promotor-driven cDNAs for mouse Kcnd2, Gabra1, Chrm1, Chrna4, Kcnj2, Kcnn1. In normal mice studied by RNA-seq, these six genes exhibited relatively higher RNA expression in 3N (spared in ALS) versus 12N and spinal cord motor neurons (vulnerable in ALS). Median survival to the time of paralysis (days) of the transgenic mice was compared to that of the control G85R SOD1YFP homozygous mice, and p-values for the plots of the transgenic strain relative to the plot of the control G85R SOD1YFP homozygous line are presented for each candidate gene.

FIG. 4 depicts crossing in a Kcnn1 transgene to the G85R SOD1YFP/G85R SOD1YFP strain to produce a G85R SOD1YFP/G85R SOD1YFP; Kcnn1/+ mouse.

FIG. 5 illustrates Kaplan-Meier survival curves displaying extended survival of hemizygous Thy1.2-Kcnn1-3; G85R SOD1YFP/G85R SOD1YFP mice as compared with their non-Kcnn1 G85R SOD1YFP/G85R SOD1YFP littermates and a cohort of G85R SOD1YFP/G85R SOD1YFP mice. The Thy1.2-Kcnn1-3 transgenic strain contains a copy number of Thy1.2-Kcnn1 of 3.0-3.8 across various mice as determined by real-time PCR of tail DNA.

FIG. 6 depicts a crossing to produce Kcnn1 homozygosity in homozygous G85R SOD1YFP/G85R SOD1YFP mice (end-product, G85R SOD1YFP/G85R SOD1YFP; Kcnn1/Kcnn1 mice).

FIG. 7 illustrates a Kaplan-Meier survival curve displaying extended survival of double homozygous Thy 1.2-Kcnn1-3/Thy1.2-Kcnn1-3; G85R SOD1YFP/G85R SOD1YFP mice as compared with a cohort of G85R SOD1YFP/G85R SOD1YFP mice. Mice containing both homozygous Kcnn1 (copy number 5.6-6.9) and G85R SOD1YFP/G85R SOD1YFP (SOD1 copy number 269-303) were followed until paralysis (n=7). The Kaplan-Meier plot of survival time for these mice was compared with a Kaplan-Meier plot of survival time of a cohort of mice (n=57) from the G85R SOD1YFP/G85R SOD1YFP colony, which were matched for SOD1 copy number.

FIG. 8 depicts crossing G93A/+; Kcnn1/+ male with Kcnn1/+ female mice to produce G93A/+; Kcnn1/Kcnn1 mice.

FIG. 9 illustrates a Kaplan-Meier survival curve displaying extended survival of homozygous Thy1.2-Kcnn1-3/Thy1.2-Kcnn1-3; G93A SOD1/+ mice (total Kcnn1 copy number 6.5-7.7) as compared with a cohort of G93A SOD1/+ mice with comparable G93A SOD1 copy numbers (190-220), both cohorts followed until paralysis. The cohort of G93A SOD1 mice (n=26) were either directly from Jackson Lab or from crosses in the laboratory that produced G93A SOD1/+ mice lacking Kcnn1

FIG. 10 illustrates a Kaplan-Meier survival curve displaying extended survival of hemizygous Thy1.2-Kcnn1-6/+; G93A/+ mice compared with a cohort of G93A SOD1/+ mice with comparable G93A SOD1 copy number (190-220). The Thy1.2-Kcnn1-6 is an independent Thy1.2-Kcnn1 transgenic line (in B6SJL background) with a hemizygous Kcnn1 copy number of 5.5-6.5. Note that the hemizygous copy number of Kcnn1-6 of the mice in this figure is somewhat lower than the homozygous Kcnn1 copy number of the mice of FIG. 9. Both strains achieve a substantial extension of survival, but the lower copy number here correlates with a somewhat reduced median survival. Consistent with such correlation, the mouse surviving 225 days here (asterix), harbored an outlying Kcnn1 copy number of 10.8.

FIG. 11 illustrates a Kaplan-Meier survival curve of two strains of mice with a lower G85R SOD1YFP total copy number ranging from 199-245 (vs 269-303 in FIG. 7). Homozygous Kcnn1-3 (total copy number here 5.6-6.9) in this setting of reduced G85R SOD1 copy number produces 100% extension of survival time (doubled), as compared with the 35% extension of survival by Kcnn1 homozygosity of the mice with G85R SOD1YFP total copy number of 269-303 in FIG. 7. This data may have implications for even stronger Kcnn1 overexpression effects on SOD1 mutant-affected humans, who have a mutant SOD1 copy number of 1.

FIG. 12 illustrates Kaplan-Meier survival curves comparing G85R SOD1YFP/G85R SOD1YFP mice (left panel); Thy1.2-Kcnn2/+; G85R SOD1YFP/G85R SOD1YFP (middle panel; with Kcnn2 copy number ~3 in one transgenic line, ~35 in a second line, and ~140 in a third line and survival times indistinguishable between the three lines, and thus combined); and Thy1.2 Kcnn1-3; G85R SOD1YFP/G85R SOD1YFP (right panel). There is no effect of Kcnn2 transgenesis, as compared with transgenesis of Kcnn1, to improve survival. (Total copy number of G85R SOD1YFP in this experiment ranged from 260-320).

FIG. 13 illustrates fluorescent images of 20 μm thick cervical spinal cord cross-sections in the ventral horn region (dotted outline) that show intact spinal cord motor neurons of long-lived Kcnn1-3/Kcnn1-3; G85R SOD1YFP/G85R SOD1YFP (doubly homozygous) mice. Top two panels are from a 9.5 month-old mouse. Middle two panels are from a 10 month-old doubly homozygous mouse. Bottom left panel shows a cervical cross-section focused on the ventral horn of a 7 month-old paralyzing G85R SOD1YFP/G85R SOD1YFP mouse. Bottom right panel shows a cervical ventral horn section from a 2 month-old G85R SOD1YFP/G85R SOD1YFP mouse with normal cell bodies in number and size, but presence of a lake-like aggregate within one neuron (arrowhead)

FIGS. 14A-B illustrate expression of Kcnn1 RNA and protein in motor neurons in ventral horn region of Kcnn1-3/Kcnn1-3 homozygous mouse and nontransgenic B6SJL as well as protein in motor neurons in ventral horn region of Kcnn1-6 and nontransgenic B6SJL. FIG. 14A top panels show RNA fluorescent in situ hybridization (FISH) of Kcnn1 RNA present in ventral horn of cervical spinal cord of a 7 month-old Kcnn1-3/Kcnn1-3 mouse (left) and a nontransgenic (B6SJL) mouse (right). Lower panels show immunostaining with an anti-Kcnn1 antibody of a Kcnn1-3/Kcnn1-3 homozygous mouse (left) and a nontransgenic B6SJL mouse (right). FIG. 14B shows immunostaining in ventral horn region with anti-Kcnn1 antibody of a Kcnn1-6/+ hemizygous mouse (left) and a nontransgenic B6SJL mouse (right).

FIG. 15 depicts Kcnn1 RNA FISH (top two panels) of motor cortex of a Kcnn1-3/Kcnn1-3 mouse (left) and a nontransgenic B6SJL mouse (right). Anti-Kcnn1 antibody staining of motor cortex of a Kcnn1-3/Kcnn1-3 homozygous mouse is shown in the panels below. At left is the same cytosolic immunostaining as observed in FIG. 14; at middle is nuclear staining of the same section with anti-Ctip2 (specific to layer V), and at right is the merge.

FIG. 16 depicts a spinal cord cross section from a 3 month old Kcnn1-3/Kcnn1-3 (5.5 copy); G85R SOD1YFP/G85R SOD1YFP (298 copy) mouse, showing a ventral horn motor neuron stained with a monoclonal antibody against Kcnn1 (left panel); localizing YFP fluorescence associated with G85R SOD1YFP protein (middle panel); and a merge of the two images (right panel). YFP fluorescence appears throughout the cytosol, with a few regions showing greater brightness. Kcnn1 exhibits a patchy pattern within the cytosol, with regions lacking signal shown to be occupied by G85R SOD1YFP in the merged image.

FIG. 17 depicts RNA expression from ~150 laser captured ventral horns from each of 3 month old B6SJL, G93A/+, and Kcnn1-6; G93A/+ mice. Equal amounts of prepared RNA were distributed to qRT/PCR with primers for the specific samples (3 from each mouse), and the amounts are expressed relative to, in the case of Kcnn1, the amount of qRT/PCR product in B6SJL, and for both mouse SOD1 and human SOD1, the amount of mouse SOD1 in B6SJL.

FIGS. 18A-B show TEM images of ventral horn motor neurons of B6SJL (control) and Kcnn1-3/Kcnn1-3 homozygous mice. FIG. 18A shows, in the top two panels, control B6SJL images at low (left) and high (right) magnification. In the upper left image, N designates nucleus; the dark bodies in the cytosol are variously lysosomes and lipofuscin granules (the latter particularly designated by a neighboring white lipid granule). In the upper right image, a tight Golgi stack is designated, G, at the left, and below it, rough ER. In the Kcnn1-3/Kcnn1-3 cell in the lower left panel, at low magnification, an expanded ER is visible in the cytoplasm and the Golgi appears dispersed into at least two locations. At right at higher magnification, another Kcnn1-3/Kcnn1-3 motor neuron shows nuclear infolds with evident cytosolic structures contained within them, as well as two MVBs (multivesicular bodies). In FIG. 18B additional images of Kcnn1-3/Kcnn1-3 motor neurons are shown. At upper left is expanded ER, at upper right is a fragmented dispersed Golgi, and at bottom left a dispersed Golgi and nuclear infold that nearly spans across the entire nucleus.

FIGS. 18C-D show TEM analyses of spinal cord motor neurons of the independent transgenic Kcnn1-6 mouse line (a mouse with Kcnn1 copy number 7.1 at 4 months of age), showing here also, in FIG. 18C left upper panel, nuclear infolds, and expanded ER and dispersed Golgi in lower panels; and in FIG. 18D expanded whorls of ER are shown. The black objects lacking surrounding membrane are lipofuscin granules, and the dark objects surrounded by membrane are lysosomes.

FIG. 19 illustrates expression from a self-complementary rAAV9 programming CFP from a CMV promoter. Virus was injected at PO into the lateral cerebral ventricles and spinal cord was taken after perfusion at 5 months of age. CFP fluorescence is evident in the majority of anti-ChAT-stained motor neurons. Shown is a cervical cord cross-section at 5 months of age.

FIG. 20 illustrates self-complementary rAAV9 genome with mouse Kcnn1 cDNA inserted downstream of CMV enhancer/promoter.

FIG. 21 shows qRT/PCR analysis of Kcnn1 RNA in spinal cord of ~135 day old mice injected ICV at PO with rAAV-CMV-Kcnn1. FIG. 21 indicates the presence of recombinant Kcnn1 RNA expressing genomes in spinal cord segments of a number of injected B6SJL or G93A/+ mice at ~135 days of age (at a time when the G93A/+ mice were paralyzing). Spinal cords were harvested, cut into ~3 mm length segments, and snap-frozen. qRT/PCR with Kcnn1 primers was then carried out, using a non-injected B6SJL mouse as control. The relative amount of product in injected vs control was then determined, and varied from ~10× to 50×. The reduced signal intensity in some of the G93A mice may relate to loss of motor neurons. Most importantly, the expression levels fell significantly short of the 100×-200× number obtained by the same procedure on constitutional Kcnn1-3/Kcnn1-3 transgenic mice.

### DETAILED DESCRIPTION

The following description and examples illustrate embodiments of the present disclosure in detail.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

The use of the singular includes the plural unless specifically stated otherwise. It must be noted that, as used in the specification, the singular forms “a,” “an” and “the” include plural referents unless the context clearly dictates otherwise.

The use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The use of “some embodiments,” “an embodiment,” “one embodiment” or “other embodiments” means that a particular feature, structure, or characteristic described in connection with the embodiments is included in at least some embodiments, but not necessarily all embodiments, of the present disclosures.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” in relation to a reference numerical value and its grammatical equivalents as used herein can include the numerical value itself and a range of values plus or minus 10% from that numerical value.

Ranges: throughout this disclosure, it should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “subject” refers to any animal, or cells thereof whether in vitro or in situ, amenable to the methods described herein. In certain non-limiting embodiments, the subject is a mammal. Exemplary subjects can be humans, apes, dogs, pigs, cattle, cats, horses, goats, sheep, rodents and other mammalians that can benefit from the therapies disclosed herein. In certain non-limiting embodiments, the patient, subject or individual is a human. Exemplary human patients can be male and/or female.

As used herein, the term “agent” refers to biologically active substances including viruses, compounds, and/or means to alter a biological mechanism. Agents can exhibit biological effects on one or more cells, tissues, circuits, or organs.

As used herein, the term “pharmaceutical composition” refers to a mixture of at least one agent useful within the present disclosure with other chemical components, such as carriers, stabilizers, diluents, dispersing agents, suspending agents, thickening agents, and/or excipients. The pharmaceutical composition facilitates administration of the agent to an organism. Multiple techniques of administering an agent or pharmaceutical composition comprising the agent exist in the art including, but not limited to: nasal, inhalational, topical, oral, buccal, rectal, pleural, peritoneal, vaginal, intramuscular, subcutaneous, transdermal, epidural, intratracheal, optic, intraocular, intracranial, intrathecal, and intravenous routes. “Parenteral” administration of an agent or pharmaceutical composition comprising the agent includes, e.g., subcutaneous (s.c.), intravenous (i.v.), intramuscular (i.m.), intrasternal injection, and/or infusion techniques.

The term “activate” as used herein, means to increase the amount of a molecule, a reaction, an interaction, a gene, an mRNA, and/or a protein's expression, stability, function, and/or activity by a measurable amount. Activators are agents that, e.g., bind to, activate, increase, speed up activation, or up-regulate a protein, a gene, or an mRNA stability, expression, function, and activity, e.g., agonists and positive allosteric modulators. Resulting activation can occur through a direct and/or indirect mechanism.

The term “inhibit” as used herein, means to reduce the amount of a molecule, a reaction, an interaction, a gene, an mRNA, and/or a protein's expression, stability, function or activity by a measurable amount or to prevent entirely. Inhibitors are agents that, e.g., bind to, partially or totally block stimulation, decrease, prevent, delay activation, inactivate, desensitize, and/or down regulate the amount of a molecule, a reaction, an interaction, a gene, an mRNA, and/or a protein's expression, stability, function, and/or activity by an amount, e.g., antagonists. Resulting inhibition can occur through a direct and/or indirect mechanism.

As used herein, the term “modulate” refers to any change in biological state, e.g., increasing, decreasing, and the like.

As used herein, the term “prevent” or “prevention” means no disorder or disease development if none had occurred, or no further disorder or disease development if there had already been development of the disorder or disease. Also considered is the ability to prevent some or all of the symptoms associated with the disorder or disease.

As used herein, “treating” or “treatment” of a state, disorder or condition includes inhibiting the state, disorder or condition, i.e., arresting or reducing the development of the disease or at least one clinical or subclinical symptom thereof, or relieving the disease, i.e., causing regression of the state, disorder or condition or at least one of its clinical or subclinical symptoms.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the subject. Some non-limiting examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the subject. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” or refers to a salt of the administered compounds prepared from pharmaceutically acceptable non-toxic acids, including inorganic acids or bases, organic acids or bases, solvates, hydrates, or clathrates thereof.

The term “effective amount” refers to a nontoxic but sufficient amount of an agent to provide the desired biological result. That result can be reduction and/or treatment of the signs, symptoms, or causes of a disease or disorder, or any other desired alteration of a biological system. An appropriate effective amount in any individual case may be determined by one of ordinary skill in the art using routine experimentation.

“Administering” is referred to herein as providing one or more pharmaceutical compositions or agents described herein to a subject. By way of example and not limitation, pharmaceutical composition or agent administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, an agent or composition of the present disclosure can comprise engineered cells or host cells expressing nucleic acid sequences described herein, or a vector comprising at least one nucleic acid sequence described herein, in an amount that is effective to treat or prevent disease. A pharmaceutical composition can comprise a target cell population as described herein, in combination with one or more pharmaceutically or physiologically acceptable carriers, diluents or excipients. Such pharmaceutical compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, viruses, and virus-like particles (eVLPs). Thus, the term “vector” includes an autonomously replicating plasmid or a virus. The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Naturally occurring” as applied to an object refers to the fact that the object can be found in nature. For example, a polypeptide or polynucleotide sequence that is present in an organism (including viruses) that can be isolated from a source in nature and which has not been intentionally modified by man is a naturally occurring sequence.

As used herein, the term “wild-type” or “normal” refers to the genotype and phenotype that is characteristic of most of the members of a species occurring naturally and contrasting with the genotype and phenotype of a mutant.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer of ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

As used herein, the term “ALS” refers to a motor neuron disease that is characterized by dysfunction and death of motor neurons that innervate voluntary striated muscle. In some embodiments, ALS is familial. In some embodiments, ALS is linked to SOD1 mutations. In other embodiments, ALS is sporadic. In other embodiments, ALS is linked to environmental factors and/or one or more genetic changes. The symptoms of ALS ameliorated by the present methods and compositions of the disclosure may include, but are not limited to, motor neuron degeneration, muscle weakness, paralysis, death, stiffness of muscles, slurred speech, aggregation of SOD1 protein, aberrations in SOD1 protein function and localization, and/or difficulty in breathing. For additional clinical description of onset, diagnosis (e.g., El Escorial criteria), progression, and management of ALS, see Amyotrophic Lateral Sclerosis, Mitsumoto, Przedborski, and Gordon eds, Taylor and Francis, New York, 2006.

As used herein, “afterhyperpolarization (AHP)” refers to the phase of an action potential when, during repolarization following the membrane potential spike, the membrane potential temporarily becomes more negative than the resting membrane potential. Briefly, the action potential spike opens calcium channels, allowing calcium entry to the cytosol. Cytosolic calcium binds to calmodulin that is stably associated with the cytosolic C-terminal tail of each subunit of a tetrameric SK channel. This binding produces an allosteric signal that opens the central pore of the SK channel (see Lee and Mackinnon, SK4, Science 2018), allowing for potassium efflux to occur that contributes to the AHP phase of the action potential.

The AHP phase modulates the firing frequency of neurons. The AHP is a primary determinant of motor neuron firing rate and changes of its duration or amplitude could alter motor neuron firing behaviors. AHP may be measured and/or estimated by methods known in the art, for example, using variability analysis of interspike intervals (ISIs).

As used herein, the term “motor coordination” refers to the orchestrated movement of multiple body parts as required to maintain posture and/or accomplish intended actions.

As used herein, the term “clearance” refers to preventing accumulation of or facilitating removal of pathogenic protein via, e.g., the endolysosomal pathway or autophagolysosomal pathway or via a Kcnn1-dependent intracellular pathway.

### II. Modifying Neurons Via Increased Kcnn1 Protein as a Means to Prevent and/or Treat Als

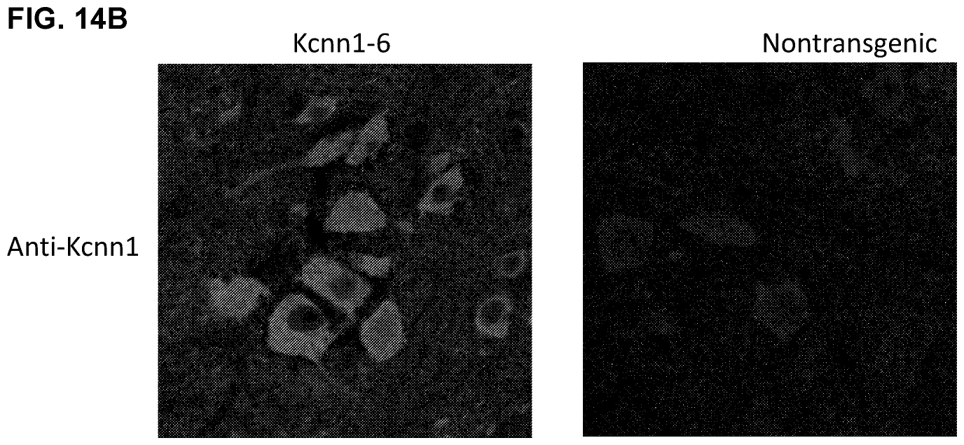

A previous study (Hadzipasic et al., PNAS 2014 111 (47) 16883-16888) showed that the fastest-firing spinal cord motor neurons are the first to die in a transgenic mouse model of ALS (homozygous G85R SOD1YFP/G85R SOD1YFP mice), suggesting that slowing motor neuron firing may potentially preserve motor neurons. Data supports that the transgenic overexpression of Kcnn1 prolongs survival, and without wishing to be defined by one theory, the therapeutic mechanism may be through decreased firing frequency of motor neurons. That is, Kcnn1 encodes a voltage-independent calcium-activated tetrameric potassium channel that is activated by the rise of intracellular calcium by an action potential, and an increase in the number of Kcnn1 channels in the setting of overexpression may downregulate neuronal excitability by elongating/deepening the afterhyperpolarization phase of an action potential where this channel is normally operative. Alternatively, overexpressed transgenic Kcnn1 subunit protein may exert, via its excess, a stress response in the cell via misfolding, misassembly, misincorporation into membranes or overoccupancy of them, and/or cytoplasmic accumulation (see FIGS. 14A-D and 16). Such stress response could lead either to reduced translation of the mutant forms of SOD1 protein, perhaps via reduction of general cap-dependent translation (e.g. as in the integrated stress response) and/or to inducing removal pathways involving autophagy or multivesicular bodies.

As used herein, “SK channels” refers to a family of four small conductance calcium activated potassium channels: Kca2.1 (SK1), KCa2.2 (SK2), Kca2.3 (SK3), Kca2.4 (SK4/IK1), which are encoded by genes Kcnn1, Kcnn2, Kcnn3, and Kcnn4, respectively. SK channels are widely expressed in neurons and are activated by an action potential-dependent increase in intracellular Ca2+ concentrations. Calcium binds to calmodulin molecules stably associated with each of the intracellular C-termini of the four SK subunits, allosterically opening the potassium channel. This contributes to the afterhyperpolarization (AHP) phase of an action potential.

Human “SK1” (KCNN1) has the following amino acid sequence
(SEQ ID NO: 1):
>sp|Q92952|KCNN1_HUMAN Small conductance calcium-activated
potassium channel protein 1 OS = Homo sapiens OX = 9606
GN = KCNN1 PE = 1 SV = 2
MNSHSYNGSVGRPLGSGPGALGRDPPDPEAGHPPQPPHSPGLQVVVAKSEPARPSPGSPR
GQPQDQDDDEDDEEDEAGRQRASGKPSNVGHRLGHRRALFEKRKRLSDYALIFGMFGIVV
MVTETELSWGVYTKESLYSFALKCLISLSTAILLGLVVLYHAREIQLFMVDNGADDWRIA
MTCERVFLISLELAVCAIHPVPGHYRFTWTARLAFTYAPSVAEADVDVLLSIPMFLRLYL
LGRVMLLHSKIFTDASSRSIGALNKITFNTRFVMKTLMTICPGTVLLVFSISSWIIAAWT
VRVCERYHDKQEVTSNFLGAMWLISITFLSIGYGDMVPHTYCGKGVCLLTGIMGAGCTAL
VVAVVARKLELTKAEKHVHNFMMDTQLTKRVKNAAANVLRETWLIYKHTRLVKKPDQARV
RKHQRKFLQAIHQAQKLRSVKIEQGKLNDQANTLTDLAKTQTVMYDLVSELHAQHEELEA
RLATLESRLDALGASLQALPGLIAQAIRPPPPPLPPRPGPGPQDQAARSSPCRWTPVAPS
DCG
The Human “SK1” (KCNN1)'s amino acid sequence is accessible
at UniProt under Q92952.
Human KCNN1 cDNA has the following nucleotide sequence
(SEQ ID NO: 2):
>NM_002248.5:323-1954 Homo sapiens potassium calcium-activated
channel subfamily N member 1 (KCNN1), transcript variant 1,
mRNA
ATGAACAGCCACAGCTACAATGGCAGCGTGGGGCGGCCGCTGGGCAGCGGGCCGGGCGCC
CTGGGACGAGACCCTCCGGACCCTGAGGCCGGCCACCCCCCACAACCCCCGCACAGCCCG
GGCCTCCAGGTGGTAGTGGCCAAGAGTGAGCCAGCCCGGCCCTCACCCGGCAGCCCCCGG
GGGCAGCCCCAGGACCAGGACGATGACGAGGATGATGAGGAAGATGAGGCCGGCAGGCAG
AGAGCCTCGGGGAAACCCTCAAATGTGGGCCACCGCCTGGGCCACCGGCGGGCGCTCTTC
GAGAAGCGGAAGCGCCTCAGCGACTATGCCCTCATTTTCGGCATGTTTGGCATCGTCGTC
ATGGTGACGGAGACCGAGCTGTCCTGGGGGGTGTACACCAAGGAGTCTCTGTACTCATTC
GCACTCAAATGCCTCATCAGCCTCTCCACGGCCATCCTGCTGGGTCTCGTTGTCCTCTAC
CATGCCCGGGAGATCCAGCTGTTCATGGTGGACAACGGGGCTGATGACTGGCGCATCGCC
ATGACCTGCGAGCGCGTGTTCCTCATCTCGCTAGAGCTGGCAGTGTGCGCCATTCACCCG
GTGCCCGGCCACTACCGCTTCACGTGGACGGCGCGGCTGGCCTTCACGTACGCGCCCTCG
GTGGCCGAGGCCGACGTGGACGTGCTGCTGTCCATCCCCATGTTCCTGCGCCTCTACCTG
CTGGGCCGGGTGATGCTACTGCACAGCAAAATCTTCACGGACGCCTCGAGCCGCAGCATC
GGGGCCCTCAACAAGATCACCTTCAACACGCGCTTCGTCATGAAGACACTCATGACCATC
TGCCCCGGCACCGTGCTGCTGGTCTTCAGCATCTCCTCCTGGATCATCGCAGCCTGGACC
GTGCGCGTCTGCGAGAGGTACCACGACAAGCAGGAAGTGACCAGCAACTTCCTGGGGGCC
ATGTGGCTGATTTCCATCACCTTCCTCTCCATTGGCTACGGCGACATGGTGCCCCACACC
TACTGCGGGAAGGGTGTGTGCCTGCTCACTGGCATCATGGGAGCTGGCTGTACCGCGCTC
GTGGTGGCTGTGGTGGCTCGGAAGCTGGAGCTCACCAAGGCTGAGAAGCACGTGCACAAC
TTCATGATGGACACTCAGCTCACCAAGCGGGTAAAAAACGCCGCTGCTAACGTTCTCAGG
GAGACGTGGCTCATCTACAAACATACCAGGCTGGTGAAGAAGCCAGACCAAGCCCGGGTT
CGGAAACACCAGCGTAAGTTCCTCCAAGCCATCCATCAGGCTCAGAAGCTCCGGAGTGTG
AAGATCGAGCAAGGGAAGCTGAACGACCAGGCTAACACGCTTACCGACCTAGCCAAGACC
CAGACCGTCATGTACGACCTTGTATCGGAGCTGCACGCTCAGCACGAGGAGCTGGAGGCC
CGCCTGGCCACCCTGGAAAGCCGCTTGGATGCGCTGGGTGCCTCTCTACAGGCCCTGCCT
GGCCTCATCGCCCAAGCCATACGCCCACCCCCGCCTCCCCTGCCTCCCAGGCCCGGCCCC
GGCCCCCAAGACCAGGCAGCCCGGAGCTCCCCCTGCCGGTGGACGCCCGTGGCCCCCTCG
GACTGCGGGTGA
The Human “SK1” (KCNN1)'s cDNA sequence is accessible at
GenBank under NM_002248.5.
Mouse “SK1” (Kcnn1) has the following amino acid sequence
(SEQ ID NO: 3)
>tr|A0A140T8Q8|A0A140T8Q8_MOUSE Small conductance calcium-
activated potassium channel protein 1 OS = Mus musculus
OX = 10090 GN = Kcnn1 PE = 4 SV = 1
MSSHSHNGSVGQPLGSGPGFLGWEPVDPEAGRPLQPTQGPGLQMVAKGQPVRLSPGGSRG
HPQEQEEEEEEEEEEEDKTGSGKPPTVSHRLGHRRALFEKRKRLSDYALIFGMFGIVVMV
TETELSWGVYTKESLCSFALKCLISLSTVILLGLVILYHAREIQLFLVDNGADDWRIAMT
WERVSLISLELVVCAIHPVPGHYRFTWTARLAFSLVPSAAEADLDVLLSIPMFLRLYLLA
RVMLLHSRIFTDASSRSIGALNRVTFNTRFVTKTLMTICPGTVLLVFSVSSWIVAAWTVR
VCERYHDKQEVTSNFLGAMWLISITFLSIGYGDMVPHTYCGKGVCLLTGIMGAGCTALVV
AVVARKLELTKAEKHVHNFMMDTQLTKRVKNAAANVLRETWLIYKHTRLVKKPDQGRVRK
HQRKFLQAIHQAQKLRSVKIEQGKVNDQANTLAELAKAQSIAYEVVSELQAQQEELEARL
AALESRLDVLGASLQALPGLIAQAICPLPPPWPGPGHLATATQSPQSHWLPTMGSDCG
Mouse “SK1” (Kcnn1) cDNA has the following nucleotide
sequence (SEQ ID NO: 4)
>NM_001363407.2: Mus musculus potassium
intermediate/small conductance calcium-activated
channel, subfamily N, member 1 (Kcnn1), transcript
variant 1, mRNA
ATGAGTAGCCACAGCCACAATGGCAGCGTGGGGCAGCCTCTGGGCAGCGGGCCTGGAT
TCCTGGGCTGGGAGCCTGTGGACCCTGAGGCAGGCCGCCCCCTGCAGCCCACCCAAGG
CCCAGGCCTGCAGATGGTGGCCAAGGGTCAGCCTGTCAGGCTGTCACCCGGTGGTTCC
AGGGGCCACCCCCAGGAGCAGGAGGAGGAAGAGGAAGAGGAGGAGGAGGAGGAGGACA
AGACAGGCTCAGGGAAGCCCCCAACAGTCAGCCACCGCCTGGGACACCGCAGGGCCCT
CTTTGAAAAGCGTAAACGGCTCAGTGACTATGCGCTCATCTTTGGCATGTTTGGGATT
GTCGTCATGGTGACAGAAACAGAGCTGTCCTGGGGTGTATACACCAAGGAGTCACTCT
GCTCTTTTGCTCTGAAATGCCTCATCAGCCTGTCCACTGTCATCTTGCTTGGCCTTGT
CATCCTGTACCACGCCCGAGAGATCCAGCTGTTCTTGGTGGACAATGGTGCCGACGAC
TGGCGTATCGCCATGACGTGGGAGCGCGTGTCCCTGATCTCGCTGGAGTTGGTCGTGT
GTGCCATCCACCCGGTGCCCGGCCACTATCGCTTCACGTGGACGGCACGACTGGCCTT
CTCTCTGGTGCCGTCGGCAGCCGAGGCAGACCTGGATGTGCTGCTGTCCATCCCCATG
TTCCTGCGCCTCTACCTGCTGGCTCGGGTCATGCTCCTGCACAGCCGCATCTTCACCG
ATGCATCCAGCCGCAGCATCGGGGCCCTTAACCGCGTCACCTTCAACACACGCTTCGT
CACCAAGACGCTCATGACCATCTGCCCCGGCACTGTGTTGTTGGTCTTCAGCGTCTCC
TCCTGGATCGTTGCTGCGTGGACAGTGCGCGTGTGTGAGAGGTACCACGATAAGCAGG
AAGTGACCAGCAACTTCCTGGGAGCCATGTGGCTCATCTCCATCACCTTCTTGTCCAT
TGGCTATGGAGACATGGTGCCGCATACCTACTGTGGGAAGGGTGTGTGTCTGCTCACT
GGCATCATGGGAGCAGGCTGCACTGCACTCGTGGTGGCCGTCGTGGCTCGGAAGTTGG
AACTCACCAAGGCTGAGAAACACGTGCACAACTTCATGATGGACACACAGCTCACCAA
GCGGGTCAAAAATGCTGCTGCAAACGTTCTCAGGGAGACATGGCTCATCTACAAACAC
ACCAGGCTGGTGAAGAAGCCAGACCAAGGCCGGGTTCGGAAACACCAGCGTAAGTTCC
TTCAGGCCATCCATCAGGCTCAGAAGCTCCGAAGTGTGAAGATTGAACAAGGGAAGGT
GAACGATCAGGCCAACACGCTGGCTGAGCTGGCCAAGGCACAGAGCATCGCATATGAG
GTGGTGTCAGAGCTGCAGGCCCAGCAGGAGGAGTTGGAGGCACGCCTAGCCGCCTTGG
AGAGCCGACTGGATGTCCTGGGTGCCTCCCTGCAGGCTCTACCAGGCCTTATAGCCCA
AGCCATATGCCCTCTACCACCACCCTGGCCTGGGCCTGGTCACCTGGCCACAGCCACC
CAGAGCCCACAAAGCCACTGGCTGCCCACCATGGGATCAGACTGTGGGTGA

In some aspects, the compositions and methods described herein comprise a polynucleotide sequence encoding the polypeptide KCNN1 of SEQ ID NO: 1 or a functional variant or fragment having at least about 80%, or at least about 90%, or at least about 95%, or at least about 97%, or at least about 99% identity thereto. In some aspects, the compositions and methods described herein comprise a polynucleotide sequence of SEQ ID NO: 2, encoding the polypeptide KCNN1 or a functional variant having at least about 80%, or at least about 90%, or at least about 95%, or at least about 97%, or at least about 99% identity thereto. In some aspects, the compositions and methods described herein comprise a codon optimized form of a polynucleotide sequence of SEQ ID NO: 2. In some aspects, the KCNN1 is a human KCNN1. In some aspects, the compositions and methods described herein comprise a polynucleotide sequence encoding the polypeptide of SEQ ID NO: 3 (mouse Kcnn1) or a functional variant or fragment having at least about 80%, or at least about 90%, or at least about 95%, or at least about 97%, or at least about 99% identity thereto. Further aspects of the compositions and methods described herein are described below. In some aspects, the compositions and methods described herein comprise a polynucleotide sequence of SEQ ID NO: 4, encoding the murine polypeptide KCNN1 or a functional variant having at least about 80%, or at least about 90%, or at least about 95%, or at least about 97%, or at least about 99% identity thereto.

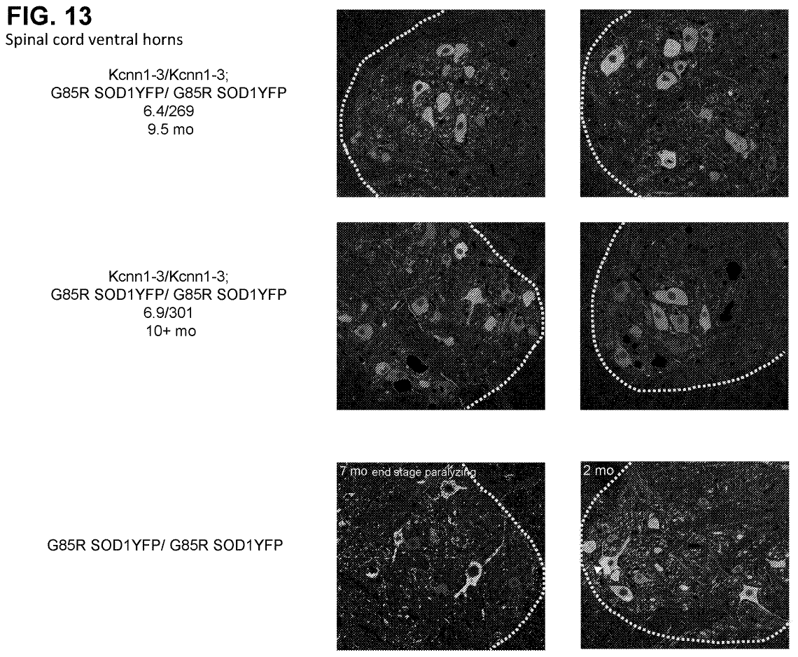

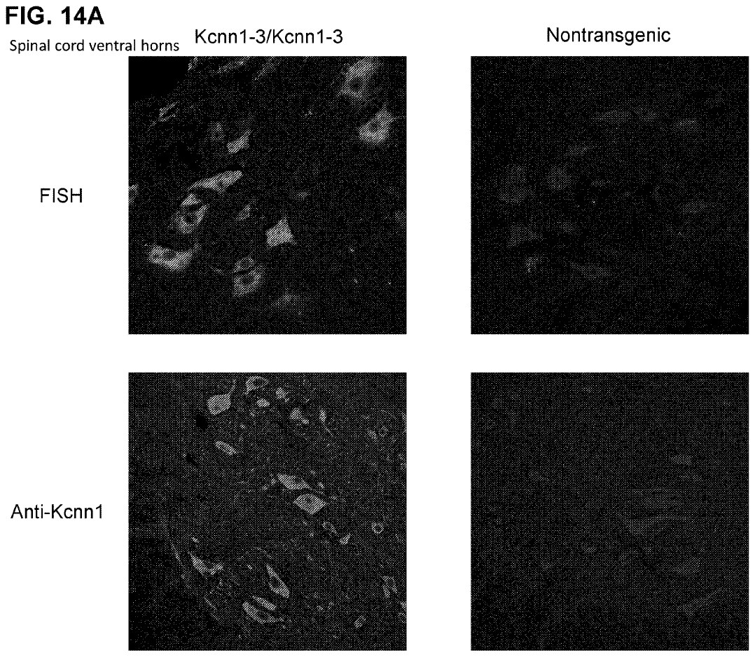

The results of Examples 1~4 support, without wishing to be defined by one theory, that a neuroprotective mechanism underlying spared (e.g., 3N) versus vulnerable (e.g., 12N or spinal cord) motor neurons in subjects with ALS may be due, at least in part, to increased Kcnn1 expression in these neurons, as indicated by RNAseq profiling. Not to be limited by one theory, transgenic overexpression of Kcnn1 may decrease firing frequency of motor neurons by producing a greater number of SK channels, producing more SK1 homotetramers, generating greater K⁺ efflux, driving a more negative membrane potential (further below resting potential), prolonging the phase of AHP, and/or a combination thereof and decreasing neuronal firing frequency. Additionally, overexpression of Kcnn1 inside of motor neurons, as shown herein (e.g., FIGS. 13-16), may be protective via a stress response involving diminished translation of or clearance of mutant SOD1 protein, effectively an induction of an intracellular action against proteotoxicity.

Thus, overexpression of Kcnn1 can lead to the increased survival of the transgenic mice (see Examples 3, 4, and 5) by any mechanism, or combination of the mechanisms, described above.

In addition to increasing SK1 channel activity by transgenic overexpression of Kcnn1, in other aspects described herein, agents that increase the expression of endogenous Kcnn1; increase the amount and/or activity of endogenous SK1 channels; increase K+ efflux; modify the AHP phase, preferably decreasing the membrane potential during AHP and/or increasing the duration of AHP; and/or decreasing and/or inhibiting firing rate in neurons (see Example 11), preferably motor neurons, prevent and/or treat ALS or are capable of treating and/or preventing ALS. Overexpression within motor neurons can also provide neuroprotective actions against neurotoxicity (see FIG. 13).

A. Agents that Increase SK1 Activity

Small molecule SK activators and/or positive modulators with varying specificity for SK1, may be used to treat and/or prevent ALS. Non-limiting examples include, but are not limited to, a triazolo pyrimidine called N-{7-[1-(4-chloro-2-methylphenoxy)ethyl]-[1,2,4]triazolo[1,5-a]pyrimidin-2-yl}-N′-methoxy-formamidine (“(-)CM-TPMF”), and derivatives; chlorzoxazone; 5,6-Dichloro-1-ethyl-1,3-dihydro-2H-benzimidazol-2-one (DCEBIO); 1-ethyl-2-benzimidazolinone (1-EBIO) and/or riluzole; or a pharmaceutically acceptable salt thereof.

(-)CM-TPMF, a potent SK1-specific activator, was reported by Hougaard et al. in 2012 (Mol. Pharm. 81, 210-219), see also (U.S. Pat. Nos. 8,765,770 and 8,685,987 B2). (-)CM-TPMF was shown to have specificity for SK1.

The activity of (-)CM-TPMF has been confirmed using HeLa cells transiently transfected with Kcnn1, measuring outward current following compound addition in patch recordings. However, the level of activity that could be attained by activating the number of channels present endogenously may not parallel the activity obtained from the increased number of channels in the setting of transgenesis.

B. Agents that Increase the Amount of Kcnn1 Protein

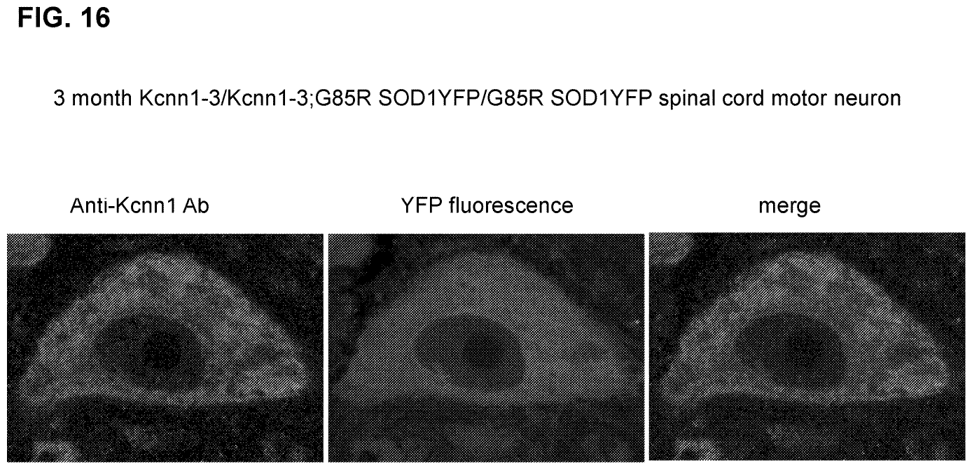

Transgenic overexpression of Kcnn1 cDNA driven by the Thy 1.2 promoter produces intracellular immunostaining of the protein at levels at least 5-10 fold greater than in non-transgenic mice (FIGS. 14A-D, 15, and 16). The intracellular pattern of staining in spinal cord motor neurons (FIG. 16) is cytoplasmic, non-uniform, and excludes the nucleus.

Without wishing to be defined by one theory, overexpression of Kcnn1 in motor neurons may clear mutant SOD1 protein and/or trigger intracellular action against proteotoxicity. Overexpressed transgenic Kcnn1 protein may exert therapeutic effects via its cytoplasmic localization, for example, by producing clearance of a toxic form(s) of the mutant cytosolic SOD1 protein or by providing additional intracellular neuroprotective effects.

### C. Gene Therapy as a Means to Increase Kcnn1 Expression and Modify SK1 Channel Activity

The nucleic acids encoding the protein(s) useful within the present disclosure, for example, SK1 channel proteins, may be used in gene therapy protocols for the treatment of the diseases or disorders contemplated herein. In certain embodiments, the diseases or disorders comprise amyotrophic lateral sclerosis. The construct encoding the protein(s) can be inserted into the appropriate gene therapy vector and administered to a subject to treat or prevent the diseases or disorder of interest.

Vectors, such as viral vectors, have been used in the prior art to introduce genes into a wide variety of different target cells. Typically, the vectors are exposed to the target cells so that transformation can take place in a sufficient proportion of the cells to provide a useful therapeutic or prophylactic effect from the expression of the desired polypeptide (e.g., a channel). The transfected nucleic acid may be retained in each of the targeted cells, providing long lasting effect, or alternatively the treatment may have to be repeated periodically.

A variety of vectors, both viral vectors and plasmid vectors are known in the art (see for example U.S. Pat. No. 5,252,479 and WO 93/07282). In particular, a number of viruses have been used as gene transfer vectors, including papovaviruses such as SV40, vaccinia virus, herpes viruses including HSV and EBV, retroviruses, and parvoviruses such as AAV. Many gene therapy protocols in the prior art have employed disabled murine retroviruses. Several issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein.

In aspects, the present disclosure provides a method for treating or preventing ALS in a subject in need thereof, comprising administering to the subject an effective amount of a pharmaceutical composition comprising a gene transfer construct, wherein the gene transfer construct comprises: (a) a human KCNN1 cDNA, or functional variant thereof having a polynucleotide sequence encoding the polypeptide of SEQ ID NO: 1 or a functional variant having at least about 95%, identity thereto; (b) an enhancer-promoter combination or a promoter operably linked to the human KCNN1 cDNA, or functional variant thereof, wherein the enhancer-promoter combination or promoter is capable of controlling expression of the human KCNN1 cDNA; (c) an inverted terminal repeat (ITR) 5′ of the enhancer-promoter combination or promoter operably linked to the human KCNN1 cDNA, or functional variant thereof; and (d) an inverted terminal repeat (ITR) 3′ of the human KCNN1 cDNA, or functional variant thereof; wherein the gene transfer construct is an adeno-associated virus 9 (AAV9). In some embodiments, the human KCNN1 cDNA comprises a polynucleotide sequence encoding the polypeptide of SEQ ID NO: 1. In some embodiments, the human KCNN1 cDNA comprises a polynucleotide sequence of SEQ ID NO: 2 or a functional variant or fragment having at least about 95% identity thereto. In some embodiments, the enhancer-promoter combination or promoter comprises a polynucleotide sequence of SEQ ID NO: 8 or a functional variant or fragment having at least about 95% identity thereto. In some embodiments, the one or more of the ITRs comprises a polynucleotide sequence of SEQ ID NO: 5, SEQ ID NO: 6, or SEQ ID NO: 7 or a functional variant or fragment having at least about 95% identity thereto.

### AAV-Mediated Gene Therapy

Adeno-associated virus (AAV), a parvovirus belonging to the genus Dependovirus, has several features that make it particularly well suited for gene therapy applications. For example, AAV can infect a wide range of host cells, including non-dividing cells. Furthermore, AAV can infect cells from a variety of species. Importantly, AAV has not been associated with any human or animal disease and does not appear to alter the physiological properties of the host cell. Finally, AAV is stable at a wide range of physical and chemical conditions, which lends itself to production, storage, and transportation requirements. For contemporary reviews, see Dunbar et al., Science 359, eaan4672, 2018, and Li and Samulski, Nature Rev Genetics 21, 255-272, 2020).

The AAV genome, a linear, single-stranded DNA molecule containing approximately 4,700 nucleotides (the AAV-2 genome consists of 4,681 nucleotides, the AAV-4 genome 4,767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome exists in an episomal form, but infectious virions are not produced. Subsequent infection by a helper virus “rescues” the episome, allowing it to be replicated and packaged into viral capsids, thereby reconstituting the infectious virion. While AAV can infect cells from different species, the helper virus must be of the same species as the host cell. Thus, for example, human AAV will replicate only in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a subject's cells are infected with the resulting rAAV virions, the heterologous nucleic acid sequence enters and is expressed in the subject's cells. Because the subject's cells lack the rep and cap genes, as well as the accessory function genes, the rAAV cannot further replicate and package genomes. Moreover, without a source of rep and cap genes, wtAAV cannot be formed in the subject's cells.

There are eleven known AAV serotypes, AAV-1 through AAV-11 (Mori, et al., 2004, Virology 330 (2): 375-83). In addition, capsid sequence variants of certain serotypes, which have different tissue or species tropism, have been produced (see, e.g., Goertsen et al., Nat Neurosci 25:106-115 (2022)). AAV-2 is the most prevalent serotype in human populations; one study estimated that at least 80% of the general population has been infected with wt AAV-2 (Berns and Linden, 1995, Bioessays 17:237-245). AAV-3 and AAV-5 are also prevalent in human populations, with infection rates of up to 60% (Georg-Fries, et al., 1984, Virology 30 134:64-71). AAV-1 and AAV-4 are simian isolates, although both serotypes can transduce human cells (Chiarini, et al., 1997, J Virol 71:6823-6833; Chou, et al., 2000, Mol Ther 2:619-623). Of the six known serotypes, AAV-2 is the best characterized. For instance, AAV-2, which has a wide tissue tropism, has been shown to transduce many different mouse and human tissue types and has been used in a broad array of in vivo transduction experiments, including in clinical contexts, hepatocytes and neurons. In the case of targeting hepatocytes, clinical benefit has resulted from recombinant AAV-mediated delivery/expression of Factor VIII or Factor IX (George et al., NEJM 385, 1961-1973, 2021; Nathwani et al, NEJM 371, 1994-2004, 2014; George et al., NEJM 377, 2215-2227, 2017). In the case of targeting neurons, clinical benefit has resulted from recombinant AAV (rAAV)-mediated delivery/expression of SMA1 via single IV dose to young children to transduce motor neurons to treat spinal muscular atrophy (Mendell et al. NEJM 377, 1713-1722, 2017) and RPE65 to retinal photoreceptors via subretinal injection of virus to treat a blindness condition (Bennett et al., Lancet, 388, 661-672, 2016). In addition, rAAV carrying expressable aromatic L-amino acid decarboxylase coding sequence has been injected into children with AADC deficiency, to either substantia nigra/ventrotegmental area (Pearson et al., Nature Comm 2021 12, Article number: 4251 (2021)) or to the putamen (Tai et al., Mol Ther 2022 Feb. 2; 30 (2): 509-518). Similarly, in Parkinson patients, the same AADC virus has been employed to enhance dopamine production in the substantia nigra (Christine et al., Ann Neurol 85, 704-714, 2019).

Delivery of a protein of interest to the cells of a mammal is accomplished by first generating an AAV vector comprising DNA encoding the protein of interest and then administering the vector to the mammal. Thus, the present disclosure should be construed to include AAV vectors comprising DNA encoding the protein of interest.

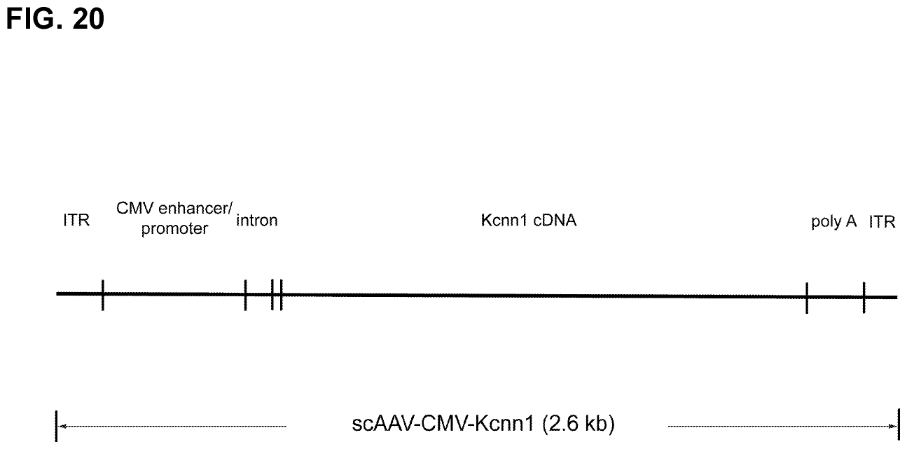

In certain embodiments, the rAAV vector comprises several DNA elements (see, e.g., FIG. 20). In certain embodiments, the construct is for use in humans. In certain embodiments, these DNA elements include at least two copies of an AAV ITR sequence (e.g., SEQ ID NO: 5 and/or SEQ ID NO: 6), a promoter/enhancer element, the sequence encoding the protein of interest, a transcription termination/polyadenylation signal, any necessary 5′ or 3′ untranslated regions which flank DNA encoding the protein of interest or a biologically active fragment thereof. In certain embodiments, one of the ITR sequences may be modified (e.g., without limitation, as in SEQ ID NO: 7) to produce a self-complementary vector. The rAAV vector of the present disclosure may also include an intron known to produce splicing. Also, optionally, the rAAV vector comprises DNA encoding a mutated protein of interest.

AAV2 ITR has the following DNA sequence
(SEQ ID NO: 5):
AGGAACCCCTAGTGATGGAGTTGGCCACTCCCTCTCTGCGCGCTCG
CTCGCTCACTGAGGCCGGGCGACCAAAGGTCGCCCGACGCCCGGGC
TTTGCCCGGGCGGCCTCAGTGAGCGAGCGAGCGCGCAG
A modified AAV2 ITR has the following DNA
sequence (SEQ ID NO: 6):
CTGCGCGCTCGCTCGCTCACTGAGGCCGCCCGGGCGTCGGGCGACC
TTTGGTCGCCCGGCCTCAGTGAGCGAGCGAGCGCGCAGAGAGGGAG
TGGCCAACTCCATCACTAGGGGTTCCT
A modified AAV2 ITR that produces self-
complementary vectors has the following
sequence (SEQ ID NO: 7):
CACTCCCTCTCTGCGCGCTCGCTCGCTCACTGAGGCCGGGCGACCA
AAGGTCGCCCGACGCCCGGGCTTTGCCCGGGCGGCCTCAGTGAGCG
AGCGAGCGCGCAGAGAGGGA

In certain aspects, one or more of the ITRs used in the compositions or methods described herein comprises a polynucleotide sequence of SEQ ID NO: 5, SEQ ID NO: 6, or SEQ ID NO: 7 or a functional variant or fragment having at least about 80%, or at least about 90%, or at least about 95%, or at least about 97%, or at least about 99% identity thereto. In some aspects, one or more of the ITRs comprises an ITR of the same serotype as the AAV capsid, comprises an ITR derived from the same serotype as the AAV capsid, comprises an ITR of a different serotype as the AAV capsid, or comprises an ITR derived from a different serotype as the AAV capsid.

In certain embodiments, the vector comprises a promoter/regulatory sequence that comprises a promiscuous promoter, which is capable of driving expression of a heterologous gene to high levels in many different cell types. Such promoters include, but are not limited to, a cytomegalovirus (CMV) enhancer/promoter, such as the cytomegalovirus (CMV) immediate early promoter/enhancer sequences (SEQ ID NO: 8), the Rous sarcoma virus promoter/enhancer sequences, a CMV enhancer fused to the chicken β-actin promoter (CAG), a chicken β-actin promoter (CBA), a simian vacuolating virus 40 (SV40) enhancer/promoter, a polyubiquitin C gene promoter (UBC), an elongation-factor 1α subunit (EF-1α) promoter, or a phosphoglycerate kinase promoter (PGK). The promoter sequence used to drive expression of the heterologous gene may be a tissue-specific promoter, such as, but not limited to, the transthyretin promoter, which is liver-specific or the muscle creatine kinase promoter/enhancer, and the like, or may be an inducible promoter, for example, but not limited to, a steroid inducible promoter.

CMV enhancer/promoter sequence (SEQ ID NO: 8):
GACATTGATTATTGACTAGTTATTAATAGTAATCAATTACGGGGTC
ATTAGTTCATAGCCCATATATGGAGTTCCGCGTTACATAACTTACG
GTAAATGGCCCGCCTGGCTGACCGCCCAACGACCCCCGCCCATTGA
CGTCAATAATGACGTATGTTCCCATAGTAACGCCAATAGGGACTTT
CCATTGACGTCAATGGGTGGAGTATTTACGGTAAACTGCCCACTTG
GCAGTACATCAAGTGTATCATATGCCAAGTACGCCCCCTATTGACG
TCAATGACGGTAAATGGCCCGCCTGGCATTATGCCCAGTACATGAC
CTTATGGGACTTTCCTACTTGGCAGTACATCTACGTATTAGTCATC
GCTATTACCATGGTGATGCGGTTTTGGCAGTACATCAATGGGCGTG
GATAGCGGTTTGACTCACGGGGATTTCCAAGTCTCCACCCCATTGA
CGTCAATGGGAGTTTGTTTTGGCACCAAAATCAACGGGACTTTCCA
AAATGTCGTAACAACTCCGCCCCATTGACGCAAATGGGCGGTAGGC
GTGTACGGTGGGAGGTCTATATAAGCAGAGCT

In certain aspects, the enhancer-promoter combination or promoter comprises a polynucleotide sequence of SEQ ID NO: 8 or a functional variant having at least about 80%, or at least about 90%, or at least about 95%, or at least about 97%, or at least about 99% identity thereto.

In some embodiments, the recombinant viral genome is self-complementary.

In certain aspects, the enhancer-promoter combination or promoter is suitable for use in humans. In certain aspects, the enhancer-promoter combination or promoter is tissue-specific, inducible, or both tissue-specific and inducible. In certain aspects, the tissue-specific enhancer-promoter combination or promoter is a neuron-specific enhancer-promoter combination or promoter. In some aspects, the neuron-specific enhancer-promoter or promoter is selected from a neuron-specific enolase (ENO2), a platelet-derived growth factor α-chain (PDGFA), a platelet-derived growth factor β-chain (PDGFB), a synapsin (SYN1), a methyl-CpG binding protein 2 (MECP2), a Ca2+/calmodulin-dependent protein kinase II (CAMK2G), metabotropic glutamate receptor 2 (GRM2), a neurofilament light (NEFL) or heavy (NEFH) chain, a proenkephalin (PENK), or an excitatory amino acid transporter 2 (SLC1A2).

In certain embodiments, the rAAV vector comprises a signal for transcription termination and polyadenylation. While any transcription termination signal may be included in the vector of the present disclosure, in certain embodiments, the transcription termination signal is the SV40 transcription termination/polyA signal. In certain embodiments, the rAAV vector comprises a post-transcriptional regulatory element (PRE). In some aspects, the gene transfer construct further comprises one or more of an intron, a transcriptional termination signal, a polyadenylation (polyA) site, an miRNA, or a post-transcriptional regulatory element (PRE). In some aspects, the gene construct comprises, from 5′ to 3′: an ITR, a CMV enhancer/promoter, an intron, a human KCNN1 cDNA, a transcriptional termination sequence, a poly A site, and an ITR.

In certain embodiments, the rAAV vector comprises isolated DNA encoding the protein of interest, or a biologically active fragment of the protein of interest. The present disclosure should be construed to include genes from mammals other than humans, which protein functions in a substantially similar manner to the human protein. Preferably, the nucleotide sequence comprising the gene encoding the protein of interest is about 50% homologous, more preferably about 70% homologous, even more preferably about 80% homologous and most preferably about 90% homologous to the gene encoding the protein of interest.

In certain embodiments, the rAAV vector is substantially in the form of FIG. 20, with the Kcnn1 cDNA being human, e.g. having the nucleotide sequence of SEQ ID NO: 2 or a variant or a fragment thereof, or encoding the amino acid sequence of SEQ ID NO: 1 or a variant or a fragment thereof.

Further, the present disclosure should be construed to include naturally occurring variants or recombinantly derived mutants of wild type protein sequences, which variants or mutants render the protein encoded thereby either as therapeutically effective as full-length protein, or even more therapeutically effective than full-length protein in the gene therapy methods as disclosed herein.

The present disclosure should also be construed to include DNA encoding variants that retain the protein's biological activity. Such variants include proteins or polypeptides that have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The present disclosure should be construed to include any suitable AAV vector, including, but not limited to, AAV1, AAV2, AAV4, AAV5, AAV6, AAV7, AAV8, or AAV9 capsid serotype, a recombinant AAV, functional variants of AAV1, AAV2, AAV4, AAV5, AAV6, AAV7, AAV8, or AAV9, including self-complementary versions thereof, and the like. Also disclosed is a method of treating a mammal having a disease or disorder with an amount effective to provide a therapeutic effect. The method comprises administering to the mammal a rAAV vector comprising the protein of interest. Preferably, the mammal is a human.

Typically, the number of viral vector genomes/mammal which are administered in a single injection ranges from about 1×10⁸ to about 5×10¹⁶. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹¹ to about 5×10¹⁴.

When the method of the present disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector in a single site injection, in certain embodiments a composition comprising the virus is injected directly into the CNS of the subject.

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

### III. Methods of Identifying or Making Als Therapies

The present disclosure also provides methods of making an ALS therapy, comprising: (a) identifying the ALS therapy by: (i) administering an effective amount of a test agent to a transgenic animal expressing G93A mutant human SOD1 or a wild-type animal; (ii) identifying the test agent as an ALS therapy if the transgenic animal demonstrates one or more of increased expression of Kcnn1, increased amount or activity of the gene product (KCNN1 protein) or of a fragment or variant thereof, improved survival time to paralysis, as compared to a control; and (b) formulating the candidate agent for administration for the treatment of ALS.

In embodiments, e.g. when overexpression of Kcnn1 confers protection via an increased amount of channel activity, there is provided a method of identifying an ALS therapy comprising: (a) identifying a candidate agent that modulates Kcnn1 channel activity comprising: (i) providing a stably transfected Kcnn1-expressing cell line or cells transiently expressing Kcnn1, (ii) treating the cells with one or more test agents, (iii) measuring a potassium export current activity (e.g. under voltage clamp conditions) that can be shown at a next level to be calcium dependent, as compared to absence of the test agent and/or compared to the positive control SK1 activator agent CM-TPMF (e.g. Hougaard et al, 2012 Mol. Pharm. 81, 210-219) (b) administering an effective amount of the candidate agent to a transgenic animal expressing G93A mutant human SOD1 or administering vehicle; (c) classifying the candidate agent as an ALS therapy if the transgenic animal demonstrates one or more of increased expression of Kcnn1, increased channel activity of Kcnn1 in spinal cord tissue slices from mice exposed to compound or vehicle (see Hadzipasic et al, PNAS, 2014 111 (47) 16883-16888), and/or improved survival time (time to paralysis), as compared to the nontreated G93A mouse; and (d) formulating the ALS therapy for administration for the treatment of ALS.

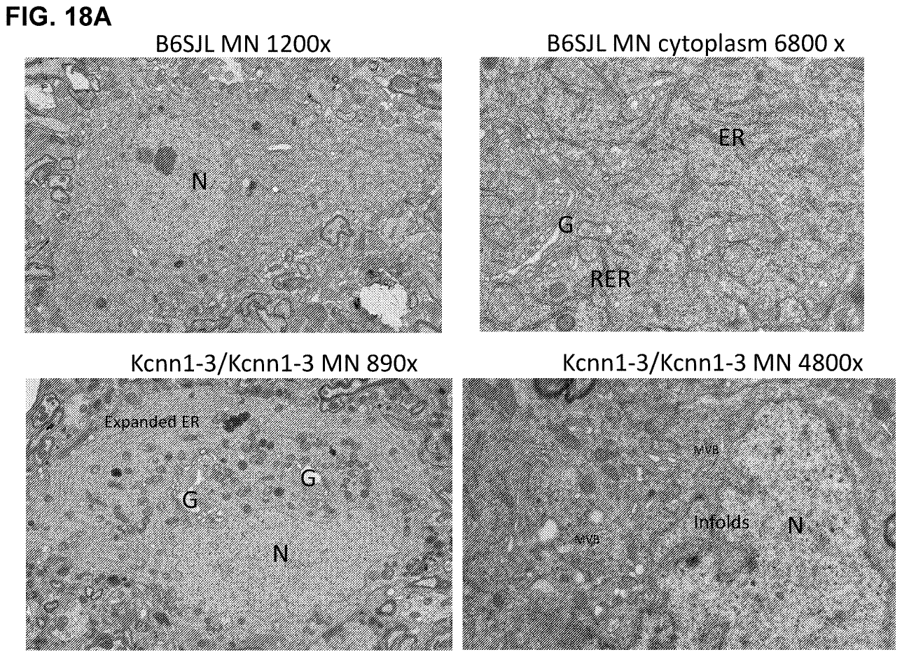

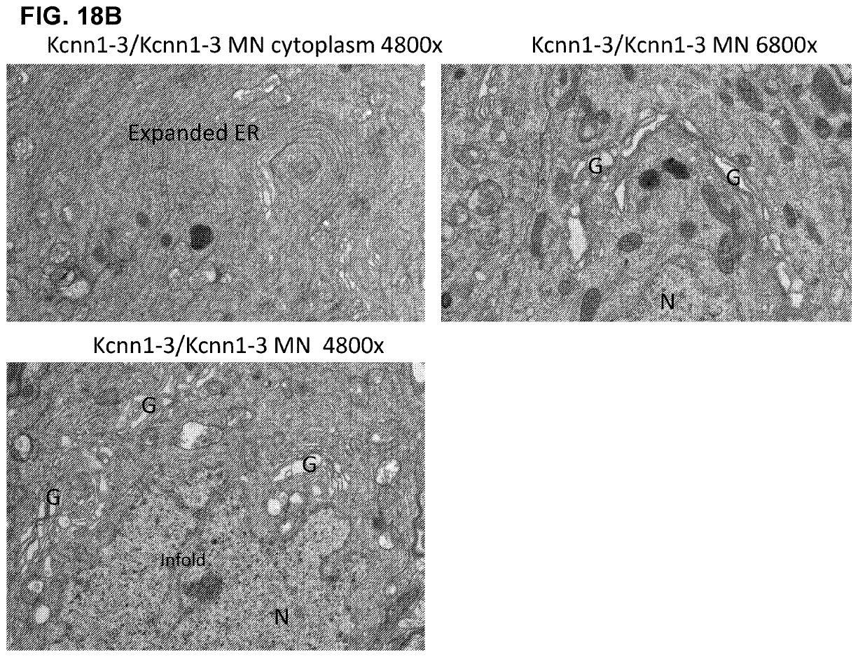

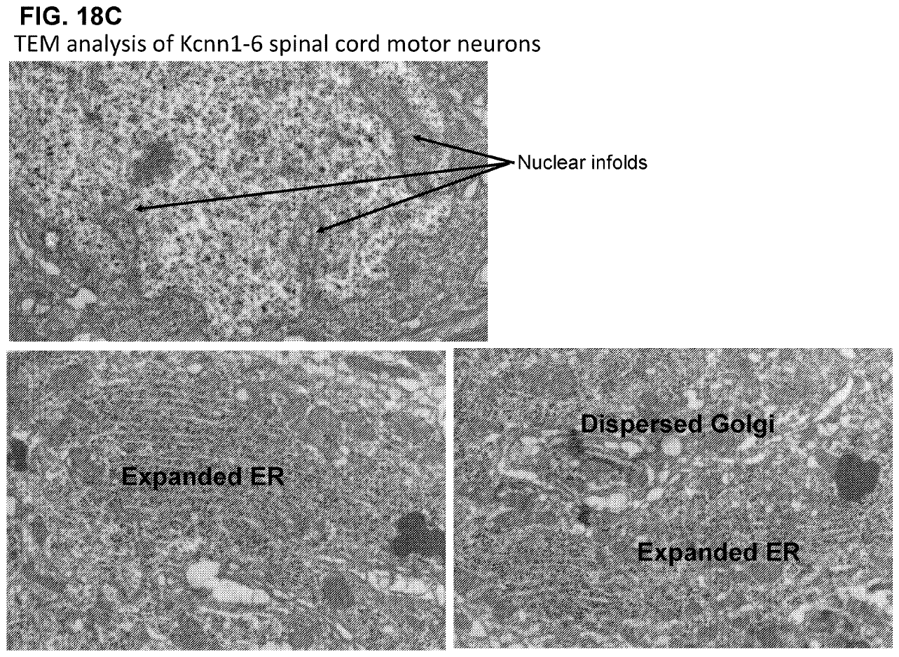

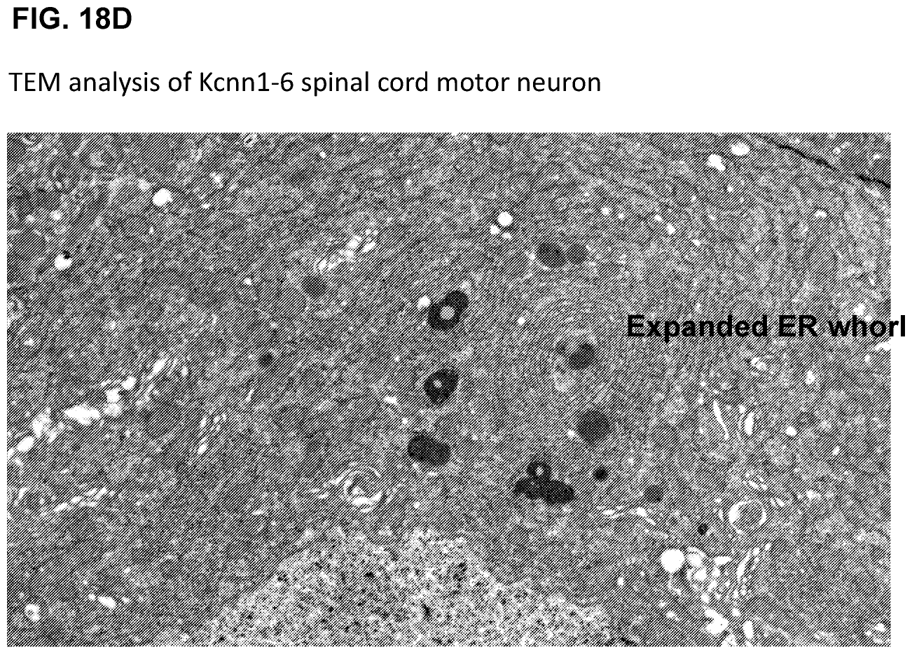

In embodiments, e.g. when overexpression of Kcnn1 confers neuroprotection via an effect of overexpression of Kcnn1 membrane protein subunit, folded and assembled or not, localized to membranes or not, effecting activation of a stress response that is protective, e.g. by induction of proteostatic pathways, which might include diminished translation or increased clearance of the proteotoxic G85R SOD1YFP or G93A SOD1, there is provided a method of identifying an ALS therapy comprising: (i) transducing cells with G85R SOD1YFP; (ii) treating the cells with chemical compounds that abolish G85R SOD1YFP fluorescence; and inspecting the cells for the same morphologic or informatic changes that are effected by Kcnn1 (see, e.g., FIGS. 18A-D for morphology changes).

The present disclosure also provides methods of identifying an ALS therapy that is effective in treating or preventing ALS comprising administering an effective amount of the agent to an SOD1-mutant mouse and determining time to paralysis, wherein an increased time to paralysis compared to an SOD1-mutant mouse that does not receive the agent indicates that the agent is an ALS therapy.

In certain aspects of the methods described herein, the ALS therapy is a gene therapy, a biologic agent, a small molecule, or a polynucleotide agent. In some aspects, the gene therapy is a viral gene therapy. In some aspects, the viral gene therapy is or comprises a recombinant adeno-associated virus (rAAV). In some aspects, the rAAV is a Kcnn1-expressing rAAV. In some aspects, the biologic agent is an antibody or peptide. In some aspects, the polynucleotide agent is selected from mRNA, siRNA, shRNA, miRNA, or cDNA.

### IV. Administration and Formulations

In certain embodiments, the agents of the present disclosure are useful in the methods described herein, when used in combination with at least one additional agent useful for treating or preventing ALS. This additional agent may comprise compounds identified herein or compounds, e.g., commercially available compounds, known to treat, prevent or reduce the symptoms of ALS in a subject.

In certain embodiments, at least one additional agent used for treating or preventing ALS comprises riluzole (Bensimon et al., 1994, New Engl. J. Med. 330:585), and is administered orally in pill form. A synergistic effect may be calculated using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect.

The regimen of administration may affect what constitutes an effective amount. The agents of the disclosure may be administered to the subject either prior to or after the onset of a disease or disorder contemplated in the present disclosure. Further, several divided dosages, as well as staggered dosages may be administered daily or sequentially, or the dose may be continuously infused, or may be a bolus injection. Further, the dosages of the agents may be proportionally increased or decreased as indicated by the exigencies of the therapeutic or prophylactic situation.

Administration of the agents of the present disclosure to a subject, preferably a mammal, more preferably a human, may be carried out using known procedures, at dosages and for periods of time effective to treat a disease or disorder contemplated in the present disclosure. An effective amount of the agent necessary to achieve a therapeutic effect may vary according to factors such as the state of the disease or disorder in the subject; the age, sex, and weight of the subject; and the ability of the agent to treat a disease or disorder contemplated in the present disclosure. Dosage regimens may be adjusted to provide the optimum therapeutic response.

Actual dosage levels of the active ingredients in the pharmaceutical compositions of the present disclosure may be varied so as to obtain an amount of the active ingredient that is effective to achieve the desired therapeutic response for a particular subject, composition, and mode of administration, without being unduly toxic to the subject.

In particular, the selected dosage level depends upon a variety of factors including the activity of the particular agent employed, the time of administration, the rate of excretion of the agent, the duration of the treatment, other drugs, compounds or materials used in combination with the agent, the age, sex, weight, condition, general health and prior medical history of the subject being treated, and like factors well-known in the medical arts.

A medical doctor, e.g., physician or veterinarian, having ordinary skill in the art may readily determine and prescribe the effective amount of the agent required. For example, the physician or veterinarian could start doses of the agent of the present disclosure employed in the pharmaceutical composition at levels lower than that required in order to achieve the desired therapeutic effect and gradually increase the dosage until the desired effect is achieved.

In particular embodiments, it is especially advantageous to formulate the agent in dosage unit form for ease of administration and uniformity of dosage. Dosage unit form as used herein refers to physically discrete units suited as unitary dosages for the subjects to be treated; each unit containing a predetermined quantity of agent calculated to produce the desired therapeutic effect in association with the required pharmaceutical vehicle. The dosage unit forms of the present disclosure are dictated by and directly dependent on (a) the unique characteristics of the agent the particular therapeutic effect to be achieved, and (b) the limitations inherent in the art of compounding/formulating such an agent for the treatment of a disease or disorder contemplated in the present disclosure.

The frequency of administration of the various combination agents of the present disclosure can vary from subject to subject depending on many factors including, but not limited to, age, disease or disorder to be treated, gender, overall health, and other factors. Thus, embodiments of the present disclosure should not be construed to be limited to any particular dosage regime and the precise dosage and composition to be administered to any subject is determined by the attending physician taking all other factors about the subject into account.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined, where desired, with other active agents.

Routes of administration of any of the agents of the present disclosure include, but are not limited to, oral, nasal, rectal, intravaginal, parenteral, buccal, sublingual, subcutaneous, intravenous, intraperitoneal, or topical. The agents for use of the present disclosure may be formulated for administration by any suitable route, such as oral or parenteral: for example, transdermal, transmucosal (e.g., sublingual, lingual, (trans) buccal, (trans) urethral, vaginal (e.g., trans- and perivaginally), (intra) nasal and (trans) rectal)), intravesicular, intrapulmonary, intraduodenal, intragastrical, intrathecal, intracerebral, intracerebellar, intracerebroventricular, subcutaneous, intramuscular, intradermal, intra-arterial, intravenous, intrabronchial, inhalation, and topical administration.

### EXAMPLES

### Example 1. Motor Neurons in the Cranial Nerve Nuclei Innervating the Extraocular Muscles of SOD1-Linked ALS Mice are Spared

Extraocular muscles are spared in all forms of human ALS, and eye movements are preserved in the later stages of the disease. The cranial nerve nuclei innervating the extraocular muscles, namely, cranial nerve nuclei 3 (3N, oculomotor), 4N (trochlear), and 6N (abducens) are likewise spared.

This Example determined whether a mouse model of ALS, bearing a high copy number (homozygous) transgene of a human genomic clone of G85R mutant SOD1 (including the human promoter) fused at its C-terminal codon with YFP (Wang, et al., 2009, Proc. Natl. Acad. Sci. USA 106:1392-1397; Hadzipasic, et al., 2014, Proc. Natl. Acad. Sci. USA 111:16883-16888) similarly exhibit sparing of motor neurons in the cranial nerve nuclei innervating the extraocular muscles.

This Example used a homozygous G85R SOD1YFP transgenic mouse model of ALS. G85R is a mutant version of SOD1 that encodes a protein that is unable to fold to an active form. A yellow fluorescent protein (YFP) reporter is attached to G85R SOD1, enabling fluorescent inspection of the mutant fusion protein in tissue. The homozygous G85R SOD1YFP transgenic mice with a transgene copy number of >260 by real-time PCR develop motor symptoms in the lower extremities by 3 months of age and become paralyzed by 6-7 months of age (see Hadzipasic et al., PNAS 2014 111 (47) 16883-16888).

At 2-3 months of age, the G85R SOD1YFP ALS mice exhibit large aggregates of the mutant fusion protein in a few spinal cord motor neurons (see FIG. 13, lower right panel, arrowhead). These are not observed at later times, presumably because these cells have been lost. Mice of this age were analyzed to determine if they exhibit aggregation in the cranial motor neurons innervating the eye. Similarly, homozygous G85R SOD1YFP mice were analyzed to determine if they exhibit aggregation in other motor neuron-containing cranial nerve nuclei, e.g., facial (7N) and hypoglossal (tongue, 12N) nuclei.

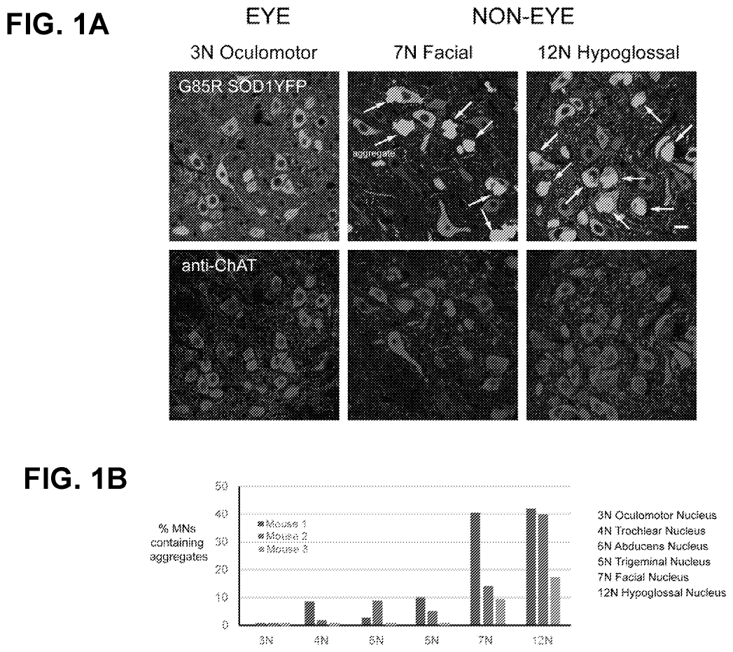

FIG. 1A shows no aggregation in motor neurons in the oculomotor nucleus (3N), relative to the significant aggregation in motor neurons of the facial (7N) and hypoglossal (12N) nuclei. In FIG. 1B (Thomas et al., BioRXiv 2018 10.1101/304857) additional quantitation of aggregates in all three extraocular cranial motor nuclei, oculomotor (3N), trochlear (4N), and abducens (6N) is compared with non-eye cranial motor nuclei, trigeminal (5N), facial (7N), and hypoglossal (12N), at 3 months of age (data from 6-7 month-old mice are similar). This further indicates that the eye-innervating motor neurons, of oculomotor (3N), trochlear (4N), and abducens (6N) nuclei, are spared relative to the non-eye cranial motor neurons in G85R SOD1YFP ALS mice.

### Example 2. Determination of Candidate Genes for Conferring Neuroprotection

Because 3N motor neurons are spared relative to 12N and spinal cord motor neurons, RNA profiling was performed from the three sites. Specifically, transcripts that are elevated in 3N relative to 12N and spinal cord, or depressed relative to 12N and spinal cord could be involved in the neuroprotection of 3N. Motor neurons were laser captured from these sites of normal wild-type mice (1,000 motor neurons from each site, 3 independent mice), and RNAseq profiles were produced from 3N, 12N, and spinal cord motor neurons.

Specifically, motor neurons in cranial nerve nuclei 3N (spared) and 12N (vulnerable) were identified by geographic position and size in Azure Blue-stained 20 μm cryostat sections of a wild-type (B6SJL) mouse brain stem. Large motor neurons in spinal cord ventral horn (vulnerable) were similarly identified. Approximately 1,000 neuron cell bodies from each nucleus and the spinal cord were laser micro-dissected as previously described (Bandyopadhyay et al, JoVE 2014; (83): 51168), total RNA was prepared (Qiagen), and polyA-selected sequencing libraries were generated from about 3 ng total RNA using a NEBNext Ultra II kit. Sequencing was performed on an Illumina NextSeq, reads were mapped and quantitated by standard techniques, and differential expression between the spared (3N) and vulnerable (12N and spinal cord) motor neurons was evaluated. Gene ontology analysis suggested that ion channels and neurotransmitter receptors were among those genes most prominently differentially expressed. Twelve genes, six over-expressed and six under-expressed in oculomotor (3N) neurons (spared) relative to hypoglossal (12N) and spinal cord motor neurons (vulnerable), were selected for transgenic overexpression or CRISPR/Cas9-mediated knockout, respectively.

### Example 3. Determination of the Neuroprotective Effects of Candidate Genes

To determine the neuroprotective effects of candidate genes, knockout and transgenic mice for candidate genes that exhibited relative decrease or increase in spared (3N) versus vulnerable motor neurons (12N and spinal cord), respectively, were produced. The knockout and transgenic lines were crossed into the homozygous G85R SOD1YFP/G85R SOD1YFP line. Mice were followed until paralysis, at which point they were euthanized, and their survival times were plotted in Kaplan-Meier curves.

Genes with relatively less expression in 3N motor neurons (spared) relative to 12N and spinal cord motor neurons (vulnerable) in normal B6SJL mice were subjected to homozygous knockout using CRISPR/Cas9: Chrm2 KO, Kcnb1 KO, Kcng1 KO, Dkk3 KO, Gabra2 KO, and Stk32a KO. Several different guide RNAs were predicted for each candidate gene, focusing on exonic regions, and were subjected to a preliminary test by electroporation into a few embryos, followed by PCR amplification and sequencing of the target regions of blastocyst DNA to determine which guides produced insertions/deletions in the most blastocysts. For each candidate gene, one or two efficient single guide RNAs were chosen and electroporated along with Cas9 protein into one-cell B6SJL embryos. Multiple founder mice for each candidate gene were identified by PCR amplification and Sanger sequencing of the target region of the candidate gene to detect and define any insertions/deletions produced. These mice were crossed to B6SJL to assess germline transmission, and transmitting female progeny were then crossed to G85R SOD1YFP homozygous males. The double hemizygous progeny (knockout/+; G85R SOD1YFP/+) were intercrossed to produce the desired progeny (knockout/knockout; G85R SOD1YFP/G85R SOD1YFP), which were scored for survival time to paralysis.

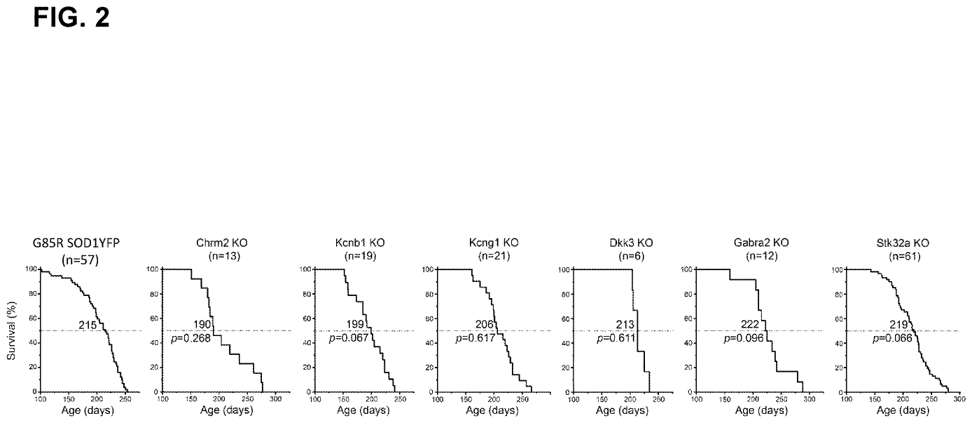

Median survival to the time of paralysis (days) of the knockout mice was compared to that of the control G85R SOD1YFP homozygous mice. Kaplan-Meier survival plots for the knockout strains and control G85R SOD1YFP homozygous mice are shown in FIG. 2. It was determined that none of the KO mice had increased or decreased survival compared to the control group.

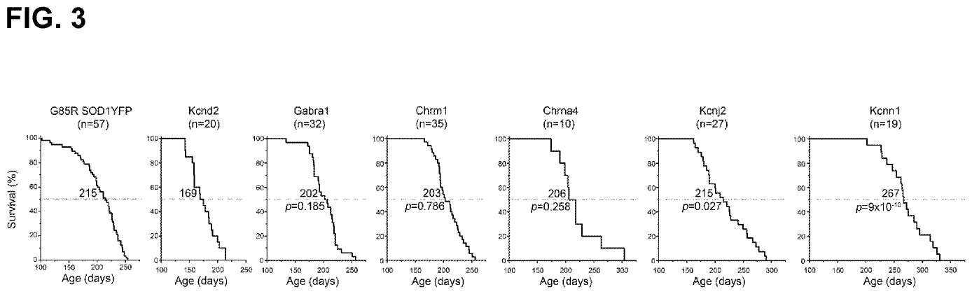

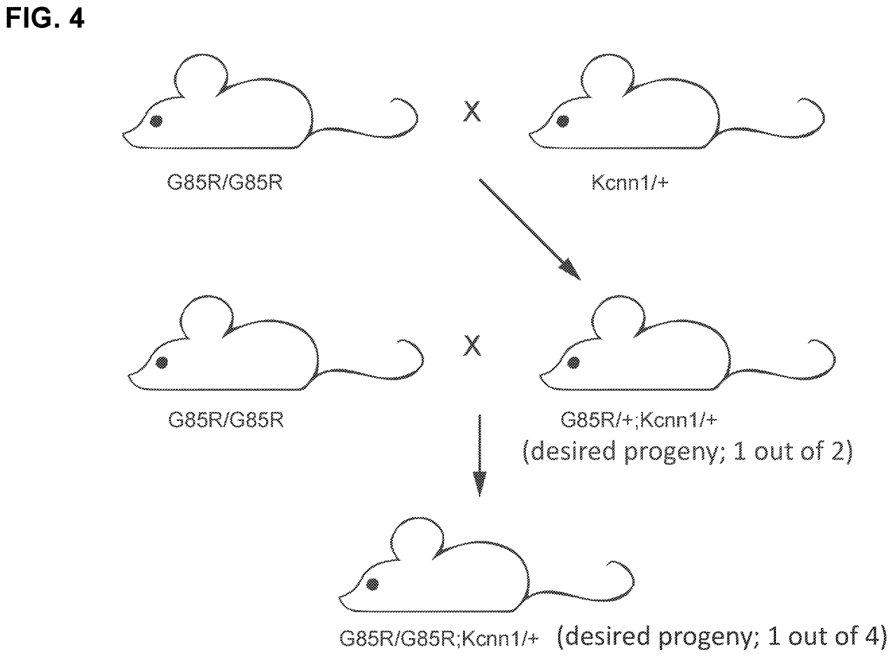

Genes with relatively more expression in 3N motor neurons (spared) relative to 12N and spinal cord motor neurons (vulnerable) were subjected to transgenesis. Hemizygous transgenic mice were produced via microinjection of one cell B6SJL embryos with linearized plasmid DNAs containing a Thy 1.2 expression cassette with inserted mouse cDNAs for the respective candidate genes (Caroni, J. Neurosci Methods 1997). The Thy 1.2 promoter favors expression in cholinergic neurons, including upper and lower motor neurons. Multiple transgenic mice with different copy number were identified for each candidate molecule by real-time PCR of tail DNA and were crossed to B6SJL to evaluate germline transmission. The transmitting mice were crossed to G85R SOD1YFP homozygous mice, and progeny hemizygous for both candidate transgene and G85R SOD1YFP were backcrossed to G85R SOD1YFP/G85R SOD1YFP to produce a hemizygous transgene of a candidate (one chromosomal array) in homozygous G85R SOD1YFP/G85R SOD1YFP (FIG. 4). Only candidate gene/+; G85R SOD1YFP/G85R SOD1YFP mice with G85R SOD1YFP copy number greater than 260 were scored for survival time to paralysis, plotted as Kaplan-Meier survival curves. In each case, several candidate gene lines with copy number across a range, which bred true in all cases, were studied. The control G85R SOD1YFP/G85R SOD1YFP strain (absent any candidate transgene) exhibited a median survival of 215 days and a maximum of 250 days. Notably, mice with the Kcnn1 transgene (hemizygous Thy1.2-Kcnn1; G85R SOD1YFP/G85R SOD1YFP) displayed a statistically significant (p=9×10-10) increase in median survival of approximately 25%. See FIG. 3.

It was determined that for the hemizygous Kcnn1 candidate gene, two transgenic lines with apparent Kcnn1 copy numbers of about 2.8 and about 3.2, produced extended survival, with genomic insertion sites determined by whole genome DNA sequencing at mouse chromosome 9 and mouse chromosome 4, respectively. The chromosome 4 site with the higher copy number was studied in detail.

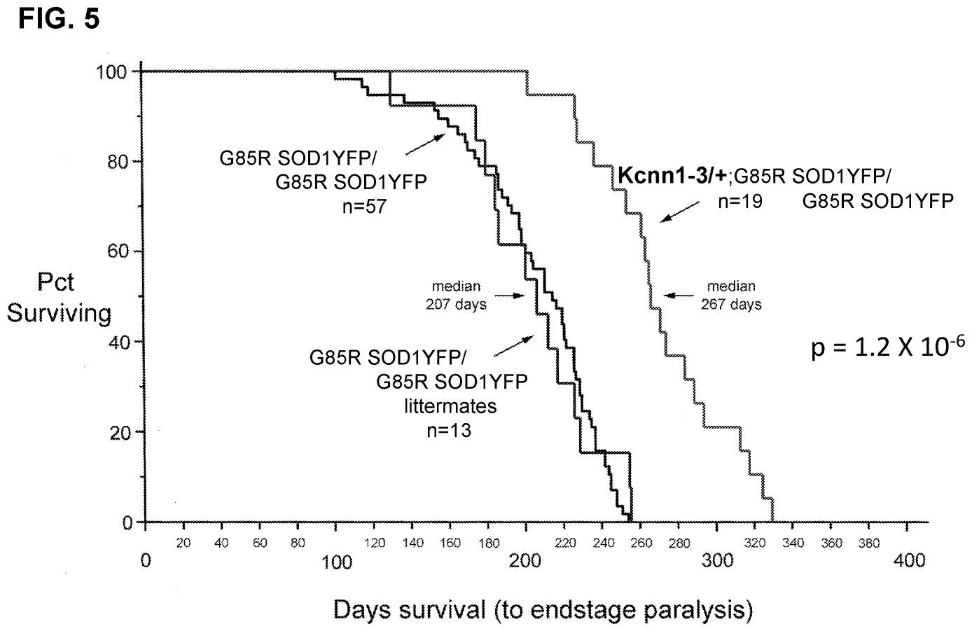

FIG. 5 shows a survival curve of mice containing both hemizygous Kcnn1 (line with copy number ~3.2, referred to as Kcnn1-3) and G85R SOD1YFP/G85R SOD1YFP (copy number >260; n=19) and of littermates lacking the Kcnn1 transgene (n=13). FIG. 5 also shows survival times of a concurrent cohort of mice from the G85R SOD1YFP/G85R SOD1YFP colony that were not otherwise involved in this experiment. Notably, the median survival of the Kcnn1-expressing mice is extended by 60 days (increased from 207 days to 267 days) compared to the littermates without the Kcnn1 transgene (p-value=1.2×10⁻⁶). The survival curve for the littermates also closely parallels that of the G85R SOD1YFP/G85R SOD1YFP colony.

### Example 4. Homozygosity of Kcnn1-3 Transgene Increases SOD1-Linked ALS Mouse Survival, Including Survival of G93A SOD1 Mice

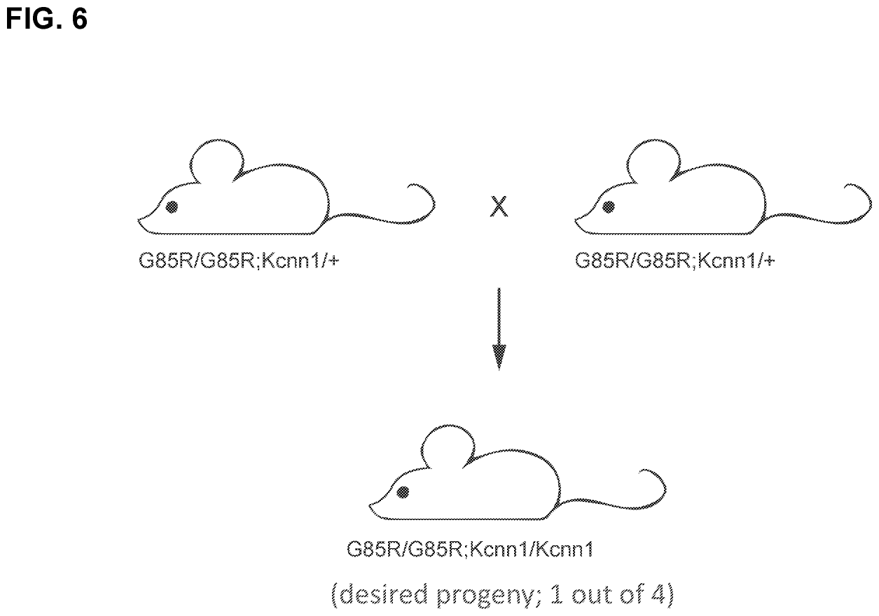

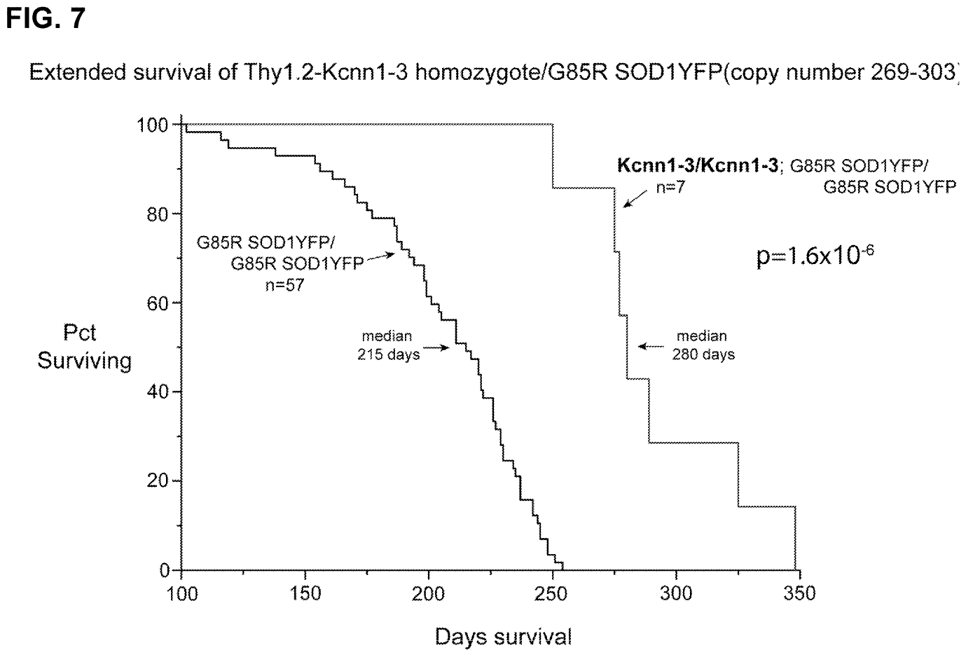

To determine whether survival of Kcnn1-3-bearing mice could be further increased by homozygosing the Thy 1.2-Kcnn1-3 transgene, Kcnn1-3/+; G85R SOD1YFP/G85R SOD1YFP mice were crossed with each other and double homozygous progeny were identified by tailing the mice and performing real time PCR. See FIG. 6. Mice containing both homozygous Kcnn1-3 (copy number 5.6-6.9) and homozygous G85R SOD1YFP (copy number 269-303) were followed until paralysis, and their survival times were plotted in Kaplan-Meier curves (FIG. 7) and were compared with Kaplan-Meier plot of survival times of the respective Kcnn1-3 hemizygous; G85R SOD1YFP/G85R SOD1YFP mice (FIG. 5). Median survival was 280 days, as compared with the median survival of 267 days, comprising a roughly 2 week increase of survival. This small extension of survival of G85R SOD1YFP homozygous mice by Kcnn1-3 homozygosis compares with a very large extension of G93A/+ survival as described below.

Males and females of hemizygous Thy 1.2-Kcnn1-3 lines without G85R SOD1YFP were also crossed and Kcnn1-3/Kcnn1-3 homozygotes with approximately double Kcnn1 copy number (5.0-6.5) as measured by real-time PCR were identified. These mice were studied, as described below, to assess the strongest effects of Kcnn1-3 on motor neurons. While Kcnn1-3 hemizygous mice were completely asymptomatic and lived to 2-3 years of age, the Kcnn1-3 homozygous mice developed varying degrees of lower extremity spasticity, as manifest when picked up by the tail, after 6-8 months of age. This behavior, not observed in Kcnn1-3/+ hemizygotes, was progressive. Despite this feature, the homozygous mice, when prodded, readily walked with a normal gait, fed normally, and females but not males were able to breed. The mice lived a normal lifespan of 2-3 years.

An independent additional strain of hemizygous Kcnn1 transgenic mice with higher copy number (~6.0) was produced, termed Kcnn1-6. The strain was crossed to hemizygosity with both G85R SOD1YFP/G85R SOD1YFP mice (progeny under study; so far, without symptoms at 6 months of age) and with the G93A SOD1/+ strain (see survival data below). The data supports the protective behavior of Kcnn1 transgenesis, with Kcnn1-6 providing a mouse line with a different insertion site (chromosome 5) and producing a copy number near to that of the homozygous Kcnn1-3 mice, supporting that transgenic copy number is the operative protective determinant. Notably the Kcnn1-6 mice do not exhibit lower extremity spasticity at later age, exhibit normal ambulation, breed normally, and survive to 2-3 years of age.

To determine whether Kcnn1 transgenesis could improve survival of the more severe SOD1-linked ALS strain, G93A SOD1/+, first Kcnn1-3 transgenic mice were mated to hemizygous G93A SOD1 (B6SJL) (Gurney) mice (B6SJL-Tg (SOD1*G93A) Gur/J) obtained from Jackson Laboratory. Mice of the G93A SOD1/+ strain (transgene copy numbers of 190-220) paralyze by ~120 days. The resulting doubly hemizygous Kcnn1-3/+; G93A SOD1/+ mice survived a median of 138 days (maximum survival 150 days) vs. a median of 124 days (maximum of 130 days) for G93A/+ mice (lacking the Kcnn1 transgene). Thus, there was an approximately 10% extension of survival conferred by transgenic Kcnn1-3 hemizygosity (data not shown).

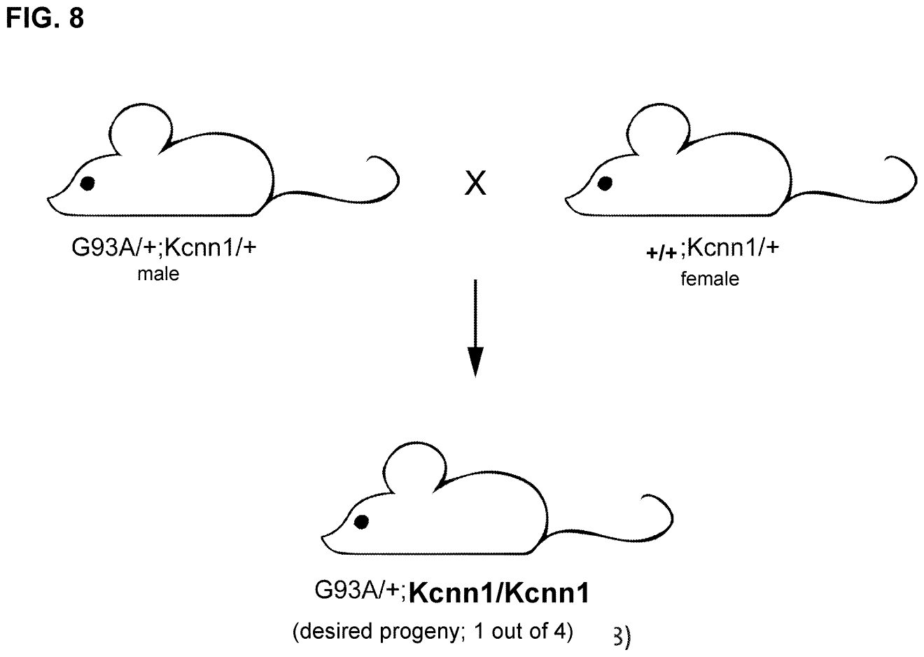

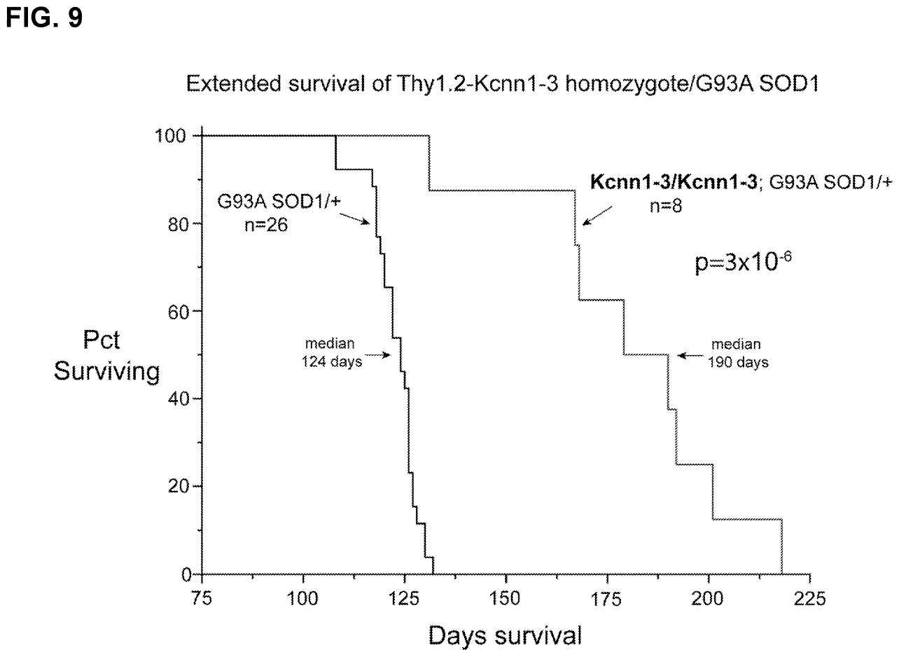

Male mice that were Kcnn1-3/+; G93A SOD1/+ were then mated with Kcnn1-3/+ female mice to produce G93A SOD1/+; Kcnn1-3/Kcnn1-3 progeny (FIG. 8). The Kcnn1-3/Kcnn1-3; G93A SOD1/+ mice exhibited substantial improvement of survival (FIG. 9). The median survival was 190 days, as compared with 124 days for the cohort of G93A SOD1 mice lacking Kcnn1. This corresponds to an extension of survival of ~2 months, or 50%. The maximum survival was 218 days, corresponding to an ~3 month (75%) extension. Thus, homozygosity for the Kcnn1-3 transgene substantially improves the survival of the G93A SOD1 mice.

Notably, the Kcnn1-3/Kcnn1-3; G93A SOD1/+ mice exhibited truncal ataxia, a “drunken sailor walk.” The Kcnn1-3/Kcnn1-3; G93A SOD1/+ mice also did not appear to “paralyze” in the same way as G93A SOD1 mice lacking Kcnn1. Instead of lower extremities held in extension, the mice were unable to get up to walk. If picked up and placed on all four extremities, the mice could ambulate across the cage, albeit with truncal ataxia. It appears that, instead of paralyzing per se, the Kcnn1-3/Kcnn1-3; G93A SOD1/+ mice progressively lose balance control, manifested as an inability to get up from an on-side position. Such behavior was apparent even before reaching endstage by their preference to sleep on-side in an empty upright dixie cup. Their overall mobility was reduced relative to increased sleeping time. For example, when offered daily wet food, they waken and move to the food. At endstage, they could no longer lift themselves to an upright position, but if assisted to rise, they were then able to walk with alternating steps over to wet food. It thus appears as if they have become unable to balance sufficiently or coordinate the necessary motor movements to get upright. Coincidingly at this stage, after taking a number of steps, they ultimately fall over to an on-side position. They are thus not classically “paralyzing” but are undergoing a progressive loss of the ability to balance. This suggested a cerebellar or vestibular effect and when anti-Kcnn1 antibody was applied to sections of deep cerebellar nuclei and vestibular nucleus of Kcnn1-3/Kcnn1-3 mice, there was significant reactivity (not shown). It appears that in the setting of G93A expression, the effects of such Kcnn1-3 expression (copy number 7-8) result in a phenotype of truncal ataxia. However, such ataxia was not observed in the standalone Kcnn1-3/Kcnn1-3 transgenics. To rule out any protective action of ataxia itself on SOD1-linked ALS, ataxic strains of mice have been crossed to G93A, and a first such strain, STX BP homozygous knockout/G93A (see Miyazaki et al, eLife 2021; 10: e59613 for STX BP knockout), has paralyzed at 120 days as would be expected for G93A alone.

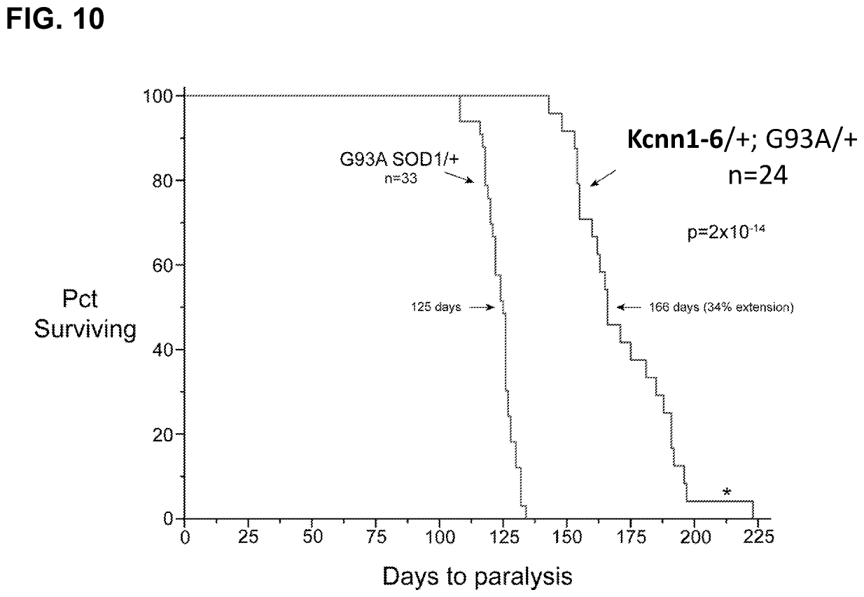

To assess whether it is increased Kcnn1 copy number that is responsible for improved survival of Kcnn1-3/Kcnn1-3; G93A/+ mice, as opposed to some action of homozygosis, e.g. insertion site effect, the higher copy hemizygous Kcnn1-6 transgenic strain of mice was crossed to G93A/+ and the double transgenic progeny scored for survival time to paralysis. FIG. 10 shows the Kaplan Meier curves for these mice as compared with G93A/+ strain. The median survival time was 166 days, comprising a 34% extension of survival. One mouse survived to 223 days but this outlier mouse harbored a Kcnn1 copy number of 10.8. Thus, considering that particular mouse as well as that the copy numbers of the Kcnn1-3 homozygous mice in FIG. 9 were 7-8 vs that in FIG. 10 being lower at ~6.0, it appears that there is a correlation of increased Kcnn1 copy number with survival of G93A SOD1 mice. Notably, the Kcnn1-6; G93A/+ mice did not exhibit signs of truncal ataxia at endstage but paralyzed with lower extremities in extension, as is usual for G93A/+ mice.

### Example 5. Reduced G85R SOD1YFP Copy Number is Associated with Profound Effect of Kcnn1-3 Homozygosis to Lengthen Survival Time to Paralysis

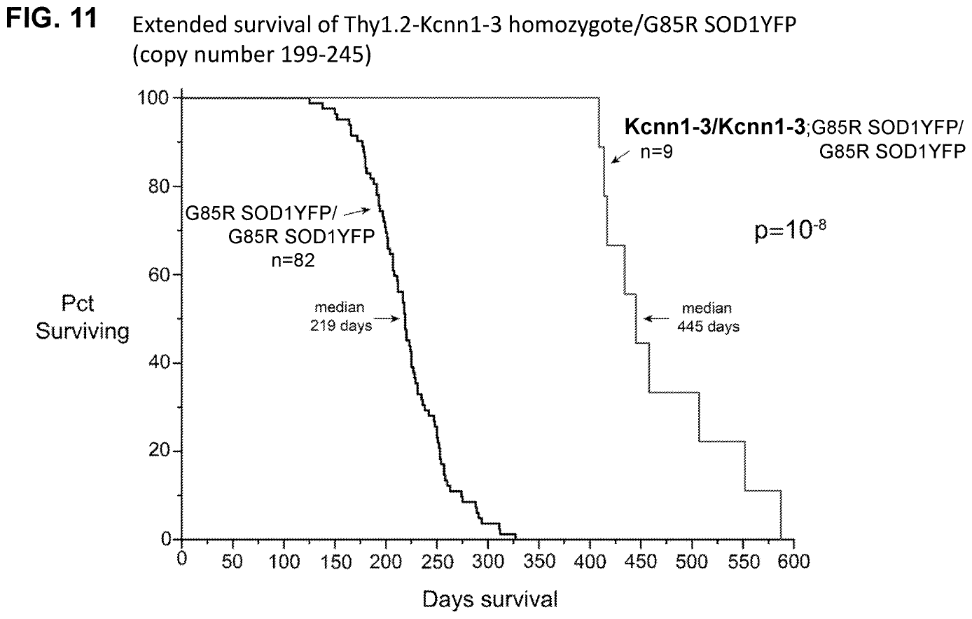

To test whether the effect of Kcnn1-3 would be more substantial in the setting of reduced G85RSOD1 copy number, Kcnn1 homozygosity (copy number ~6.0) was bred into a cohort of G85R SOD1YFP/G85R SOD1YFP mice with a lower copy number ranging from 199-245 (vs. 269-303 in FIG. 7). There was a remarkable effect on survival (FIG. 11), with the double homozygous mice all living well beyond a year and with a median survival of 445 days compared with the median of 219 days for the copy-matched G85R SOD1YFP homozygotes. This amounted to a doubling of survival, and speaks to the more potent effect of Kcnn1 transgenesis in a lower G85R SOD1YFP copy number setting. It speaks also to the possibly even larger potency in the context of the human setting where the copy number of mutant SOD1 equals one. As regards the human single copy situation, the need for homozygous Kcnn1, with some attendant symptoms after a period of time, might be obviated, and the use of low copy Kcnn1 (e.g. copy number 3-3.5 in the original heterozygous Kcnn1-3/+; G85R SOD1YFP/G85R SOD1YFP, see FIG. 5), did not confer symptoms of spasticity or ataxia to transgenic mice and might be feasible for long-term survival.

### Example 6. The Extended Survival Mediated by Kcnn1 Transgenesis is not Reproduced by Transgenesis with Kcnn2

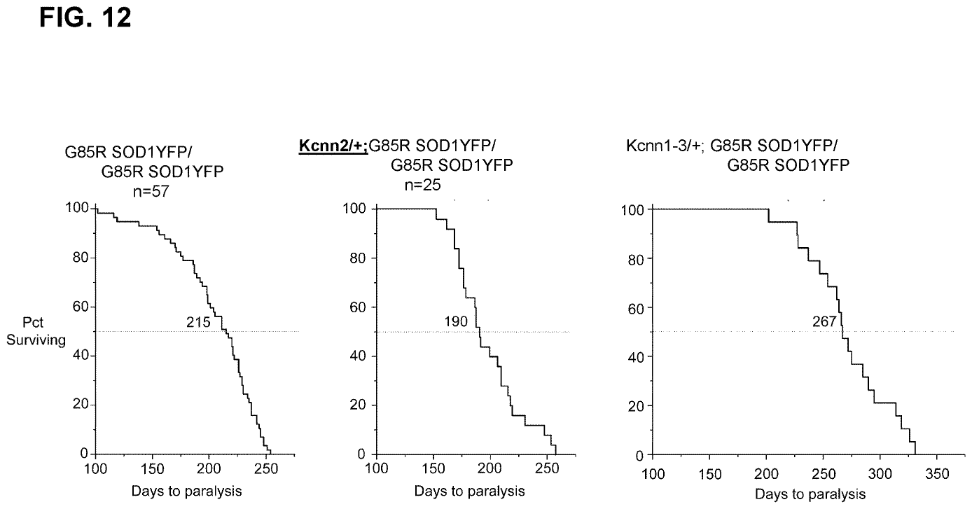

To test whether Kcnn2, encoding another SK channel subunit that is found in motor neurons, can also mediate protection from G85R SOD1YFP, three transgenic lines of Thy 1.2-Kcnn2 were produced, with hemizygous copy numbers of ~3, ~30, and ~150, and these lines were crossed with G85R SOD1YFP as in FIG. 4 to produce Kcnn2/+; G85R SOD1YFP/G85R SOD1YFP mice that were scored for survival time to paralysis. As shown in FIG. 12, there was no extension of survival observed relative to the G85R SOD1YFP/G85R SOD1YFP and Kcnn1; G85R SOD1YFP/G85R SOD1YFP strains. At a further level, the three individual Kcnn2; G85RSOD1YFP/G85R SOD1YFP strains exhibited indistinguishable survival times and were thus combined in the plot that is shown. Thus the beneficial effects of Kcnn1 appear to be specific and may relate to its N-terminal cytosolic region, which has an entirely different amino acid sequence (89 amino acids) than that of Kcnn2 (382 amino acids), whereas the remainder of the two proteins through the channel portion and calmodulin binding portion is highly-conserved, with the C-terminal penultimate 50 amino acids modestly conserved and last 25 amino acids not conserved. Preliminarily, fusion of the Kcnn1 N-terminus to the remainder of Kcnn2 appears to be able to confer extension of survival of G85R SOD1/G85R SOD1 mice.

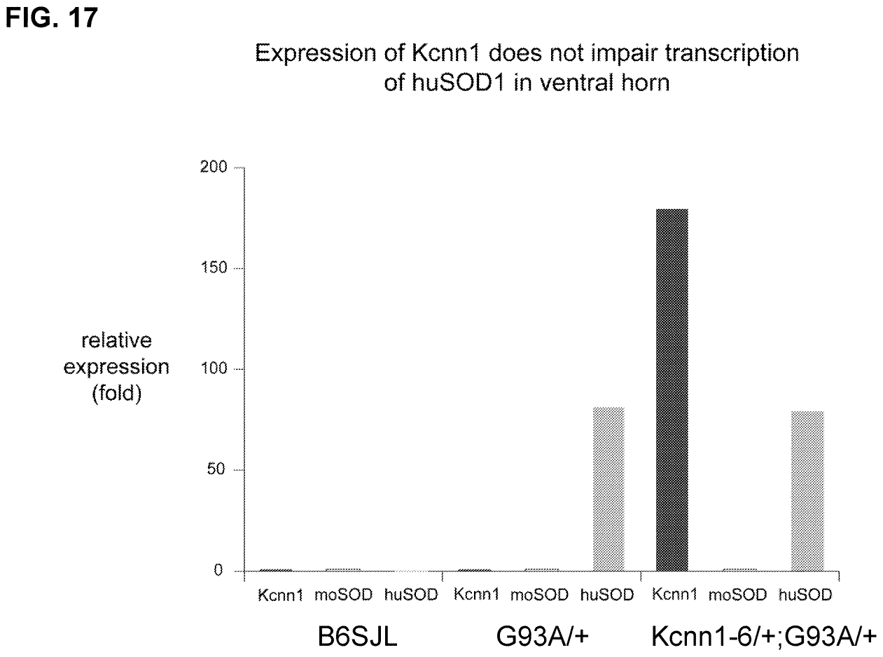

To determine if Kcnn1 transgenesis is affecting expression or stability of mutant SOD1 in Kcnn1-3/+; G85R SOD1YFP/G85R SOD1YFP mice, YFP fluorescence in their spinal cord motor neurons was compared with that in similar neurons in age-matched and copy number-matched G85R SOD1YFP/G85R SOD1YFP mice. This study showed no difference in the distribution of spinal cord motor neuron fluorescence intensity, suggesting that transcription and translation of G85R SOD1YFP are not affected by Kcnn1 expression (see FIG. 17 concerning direct transcriptional measurements). Similarly, the amounts of transgenic SOD1 RNA in brains of G85R SOD1YFP/G85R SOD1YFP or G93A SOD1/+ mice were not affected by the presence of either hemizygous or homozygous Kcnn1 transgenes (not shown), supporting that transcription is unaffected. Thus, a post-translational effect of Kcnn1 expression appears to be operating.

Overall, transgenic overexpression of Kcnn1 could, without wishing to be defined by one theory, produce more SK channels resulting in prolonged afterhyperpolarization and decreased firing frequency of motor neurons, protecting them, or, alternatively, overexpression of the Kcnn1 subunit may produce an intracellular effect, e.g. via accretion in membranes or in the cytosol, or in both locations, that counters the toxicity of mutant SOD1, e.g., without wishing to be defined by one theory, via an induced clearance mechanism(s). Either mechanism, each dependent on overexpression of Kcnn1, affords a means of treatment of ALS.

### Example 7. Retention of Spinal Cord Motor Neuron Morphology is Associated with the Improved Survival in the Setting of Transgenic Kcnn1 Overexpression

The improvement in survival of G85R SOD1YFP mice containing homozygous Kcnn1-3 was shown to be associated with retention of normal morphology of the spinal cord motor neurons and absence of YFP-fluorescent aggregates.

Two homozygous Kcnn1-3/Kcnn1-3 mice (with G85R SOD1YFP copy numbers of 269 and 301), were perfused at 9.5 and 10.5 months of age. Spinal cords were extracted and examined for G85R SOD1YFP fluorescence (FIG. 13). Specifically, the top two panels are from a 9.5 month-old doubly homozygous mouse with a total Kcnn1 copy number of 6.4 and a G85R SOD1YFP copy number of 269. The middle two panels show similar sections from a 10 month-old doubly homozygous mouse with a Kcnn1 copy number of 6.9 and a G85R SOD1YFP copy number of 301. The lower left panel shows a cervical cross-section focused on the ventral horn of a 7 month-old paralyzing G85R SOD1YFP/G85R SOD1YFP mouse. There is a reduced number of intact motor neuron cell bodies, irregular fluorescence of the remaining neurons, and the presence of fragments of YFP-fluorescent material in the neuropil. The lower right panel shows a cervical ventral horn section from a 2 month-old G85R SOD1YFP/G85R SOD1YFP mouse with normal cell bodies in number and size. A single lake-like aggregate is present in one motor neuron, marked by an arrowhead.

The improvement in survival and retention of normal spinal cord morphology also correlated with strong expression of Kcnn1, as determined both by RNA FISH and by immunostaining with anti-Kcnn1 antibodies (FIGS. 14A-B). FISH was carried out using the Stellaris platform and produced strong signals in spinal cord motor neurons of a Kcnn1-3/Kcnn1-3 mouse (left upper panel) as compared to a non-transgenic B6SJL mouse (right upper panel). The signal was at least 10-fold greater. This finding is consistent with an 80-fold increase in Kcnn1 RNA following a comparison of total brain RNA using qRT-PCR. When anti-Kcnn1 antibody staining was examined, the difference was approximately 10-fold (compare left with right lower panels). Immunostaining with anti-Kcnn1 was also carried out on ventral horn from a hemizygous Kcnn1-6 mouse and non-transgenic B6SJL in a parallel experiment (FIG. 14B), and produced a similar large increase of anti-Kcnn1 staining in the transgenic motor neurons.

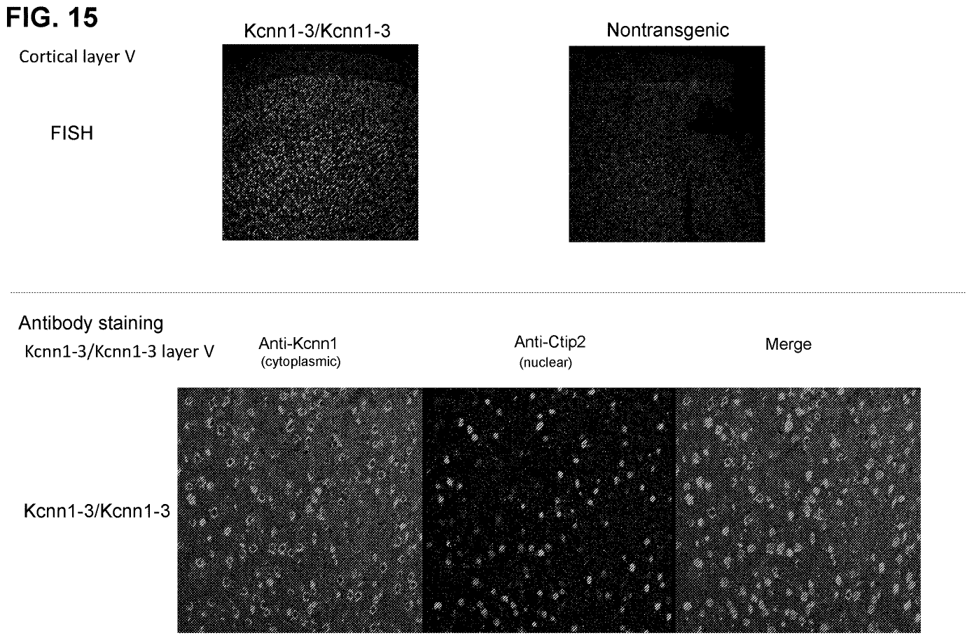

Similar strong Kcnn1 RNA expression was observed for upper motor neurons when motor cortex layer V of a Kcnn1-3/Kcnn1-3 transgenic mouse was examined by FISH (FIG. 15, top panels). Immunostaining with anti-Kcnn1 monoclonal antibody revealed strong signals in layer V in the cytosol of many CTIP2-positive motor neurons, resembling the cytosolic antibody staining observed in ventral horn motor neurons (see FIGS. 14A-B), which supports that Kcnn1 likely exerts favorable effects on upper motor neurons, as well as on spinal cord motor neurons.

### Example 8. Localization of Kcnn1 Relative to G85R SOD1YFP in Cytoplasm of Motor Neurons

Kcnn1 and G85R SOD1YFP protein expression were assessed in cervical ventral horn motor neurons of a 3 month old mouse with genotype Kcnn1-3/Kcnn1-3; G85R SOD1YFP/G85R SOD1YFP, with copy numbers of 5.5 and 298, respectively. FIG. 16 shows 100× confocal images of a single motor neuron. By anti-Kcnn1 antibody-staining (left panel), it is apparent that Kcnn1 is strongly localized in the cytoplasm in a non-uniform pattern and excluded from the nucleus. Intracytoplasmic staining appears to be non-regular; specifically, there are non-staining “patches” in the cytoplasm. This could be an artefact of the requirement for a preparation protocol that involves rapid freezing/embedding of freshly dissected spinal cord tissue in OCT, followed by rapid “post-fixation” of cut sections following cryostat sectioning. To date, it has not been possible to observe colocalization of anti-Kenn-positive regions with specific structures, and it may be that cytosolic multimerization/aggregation of overexpressed Kcnn1 is occurring in the cytoplasm in a patchy manner. Note in the merged image that regions not occupied with anti-Kenn signal contain G85R SOD1YFP fluorescence, which is more uniform in the standalone middle panel.

Note that the localization of Kcnn1 observed here suggests that overexpressed Kcnn1 is localizing in sites other than the plasma membrane, where channel activity would be thought to occur. For example, without wishing to be bound by theory, cytoplasmic Kcnn1 protein may contribute to a response that produces clearance of mutant SOD1 protein, hence reducing its toxicity.

### Example 9. Effects of Kcnn1 are Post-Transcriptional: Kcnn1 does not Impair Transcription of huSOD1 in Ventral Horns of the Spinal Cord

Approximately 100 ventral horns were laser capture microdissected from each of a 3 month B6SJL mouse, a 3 month G93A/+ mouse, and a Kcnn1-6; G93A/+ mouse. RNA was prepared and subjected in each case to qRT/PCR of Kcnn1, mouse SOD1, and human SOD1. Kcnn1 expression was measured relative to that of the B6SJL mouse, and both moSOD1 and huSOD1 levels were measured relative to endogenous mouse SOD1 from B6SJL (FIG. 17). It is apparent that Kcnn1 transgenesis in the Kcnn1-6; G93A/+ mouse does not affect the level of transcription of human G93A SOD1 RNA (compare green bar with that of G93A alone). Thus the effects in motor neurons in vivo of Kcnn1 expression to improve survival are apparently not an effect of reducing transcription of the mutant SOD1 RNA species, and the effect(s) of Kcnn1 transgenesis must lie at a post-transcriptional level.

### Example 10. a Stress-Like State May be Established by Kcnn1, which Protects Motor Neurons from Effects of Mutant SOD1

FIGS. 18A-18D show TEM analyses of ventral horn motor neurons from B6SJL (control), Kcnn1-3/Kcnn1-3 homozygous mice, and hemizygous Kcnn1-6 mice at 3 months of age. Several prominent abnormalities are observed, each recurring in many neurons in the Kcnn1-3/Kcnn1-3 and Kcnn1-6 mice:
1) The nuclear envelope exhibits infolds (FIG. 18A lower right panel and top panel FIG. 18C), with cytosolic contents, e.g. ribosomes present in the infold, reflecting that infolding involves both leaflets of the nuclear envelope (see Malhas et al, Trends in Cell Biol 2011 Volume 21, Issue 6, June 2011, Pages 362-373; formation of such infolds has been associated with heat shock).
2) The ER exhibits expansion into parallel stacks that appear to represent an increased amount of ER production (see FIG. 18B upper left and FIG. 18C and FIG. 18D). Some of the expanded ER appears to be devoid of ribosomes whereas other portions are rough ER, studded with ribosomes.
3) The Golgi cisternae are dispersed instead of tightly stacked (FIG. 18B, upper right and lower left panels and FIG. 18C), and in some cases the cisternae appear dilated. This was visible also in confocal microscopy with anti-GM130 antibody (not shown).
4) There appears to be an increase in the number of multivesicular bodies, as marked MVB in FIG. 18A lower right panel.

### Example 11. Assessment of Kcnn1 rAAV Transgenesis

Based on the results demonstrating that Kcnn1 transgene hemizygosity and homozygosity increase ALS mouse survival, tests were undertaken to determine whether infection with recombinant AAVs encoding Kcnn1 protein can increase survival in mouse models of ALS.

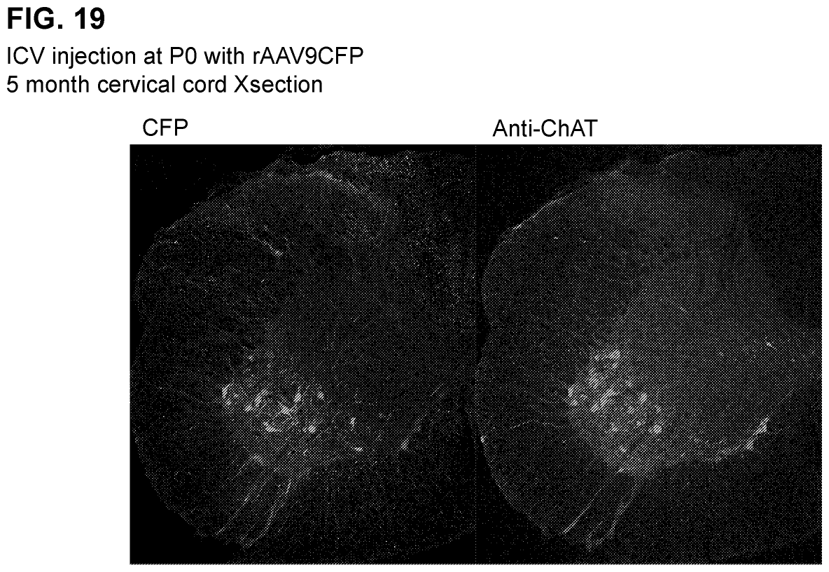

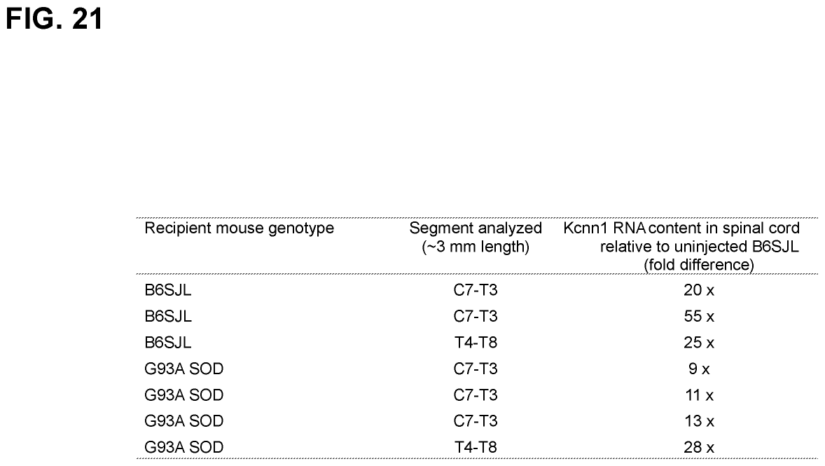

Specifically, the effects of such an AAV on survival time are investigated in the context of long-term expression following intracerebroventricular injection (ICV injection) of recombinant self-complementary AAV9-CMV-Kcnn1 at day PO (within the first 8 hr of birth). Such injection of mouse pups has been recognized to efficiently transduce motor neurons, and this was confirmed using a self-complementary AAV9 carrying a CMV enhancer/promoter driving CFP (see FIG. 19). At 5 months of age expression of bright CFP fluorescence in most ChAT-positive motor neurons of the ventral horn was observed. Accordingly, a similar self-complementary virus was constructed using the same promoter system to drive Kcnn1 cDNA (FIG. 20). A titer of 1.6×10E13 particles/ml was achieved at the packaging step and virus was injected bilaterally ICV into PO pups from a cross of B6SJL with G93A/+. Later genotyping revealed the expected 50:50 distribution of G93A vs B6SJL progeny. Both sets of mice were observed and, excitingly, the G93A mice passed 124 days, our median for survival, without significant motor symptoms. However, they began to lose weight and were all paralyzed by 132-140 days, representing only a small gain of survival. To establish that the mice had been transduced, qRT/PCR was carried out on freshly frozen spinal cord tissue segments and it was observed that there was indeed significant expression of Kcnn1 RNA in both injected B6SJL mice and injected G93A mice, amounting to anywhere from 9-fold to 55-fold that of an uninjected B6SJL mouse (FIG. 21). This was well correlated with the extension of survival. That is, the level of RNA from cord lay well short of the expression level of Kcnn1-3/Kcnn1-3 transgenic G93A mice, where levels of Kcnn1 RNA prepared from spinal cord by the same means and assessed by qRT/PCR were 100-200× that of non-transgenic B6SJL. Notably, anti-Kcnn1 antibody staining of cord sections from the virus-injected mice was also carried out and a small signal from motor neurons, much weaker signal than that of transgenic Kcnn1-3/Kcnn1-3 mice but noticeably greater than that of nontransgenic B6SJL mice was observed (not shown). We concluded that a higher titer of virus could be most immediately helpful to expression level and survival and prepared a titer of the same AAV9 Kcnn1 virus at >4×10E13 and injected ~20 PO pups.

### Example 12. Assessment of a Putative Stress Response Induced in Motor Neurons Overexpressing Kcnn1; the Soluble/Insoluble State and Monomer Vs Tetramer State of Overexpressed Kcnn1 Protein; Physical Association of Overexpressed Kcnn1 Protein with Other Proteins; Ability of Channel-Defective State to Maintain Improvement of Survival; and Assessment of Late Onset of Kcnn1 Expression on ALS Survival Time

Experiments will assess first whether motor neurons in transgenic Kcnn1-ovexpressing mice undergo a stress response that is conferring protection from toxicity of mutant SOD1 and enabling extended survival. This will be accomplished using laser capture microdissection of 1000-2000 motor neurons from Azure B-stained fresh frozen cord sections of Kcnn1-3/Kcnn1-3, Kcnn1-6, and B6SJL control mice, followed by RNAseq which will assess the entire respective transcriptomes. Preliminary testing of several stress response genes using ~100 laser captured whole ventral horns has indicated induction of several stress response RNAs in the Kcnn1-3/Kcnn1-3 spinal cords. Direct study of motor neurons should fully resolve whether these components are involved, as well as reveal any other components or pathways that are producing protection.

Western blot analysis of fractionated spinal cord or brain extracts from transgenic Kcnn1 strains should allow assessment of whether the Kcnn1 protein observed by immunofluorescence is membrane-associated, as might be expected given presence of the transmembrane domains, or soluble, potentially as some form of oligomer. Immunoprecipitation from solubilized extracts may allow detection, by mass spectrometry, of other proteins that become associated with overexpressed Kcnn1, e.g. heat shock proteins or misfolded SOD1 itself.

To formally assess whether a form of Kcnn1 without channel activity can still improve survival of SOD1-mutant strains, a Thy 1.2-Kenn mutant transgene has been designed that contains a double substitution, R464E; K467E in helix HB, that abolishes calmodulin binding to the C-terminal region of Kcnn1 subunits and thus abolishes ability of calcium to open the channel. If channel activity is not required, the extended survival of overexpressing the mutant Kcnn1 should be retained.

We also wish to assess whether late onset expression of Kcnn1 can provide improved survival of G85R SOD1YFP/G85R SOD1YFP mice or G93A SOD1/+ mice. We are attempting to use various inducible systems that can induce Kcnn1 in motor neurons by administration or withdrawal of doxycycline, but have not so far been able to generate the correct triple transgenic mice to carry out a test. However, given the late onset observed in human subjects with ALS, the ability to “rescue” already-clinically observable ALS is of importance.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. All publications, patent applications, patents, and other references mentioned herein are incorporated by reference in their entirety.

What is claimed is:

1. A composition comprising a gene transfer construct, wherein the gene transfer construct comprises:
(a) a human KCNN1 cDNA, or functional variant thereof;
(b) an enhancer-promoter combination or a promoter operably linked to the human KCNN1 cDNA, or functional variant thereof, wherein the enhancer-promoter combination or promoter is capable of controlling expression of the human KCNN1 cDNA;
(c) an inverted terminal repeat (ITR) 5′ of the enhancer-promoter combination or promoter operably linked to the human KCNN1 cDNA, or functional variant thereof; and
(d) an inverted terminal repeat (ITR) 3′ of the human KCNN1 cDNA, or functional variant thereof.

2. The composition of claim 1, wherein the gene transfer construct is an adeno-associated virus (AAV).

3. The composition of claim 2, wherein the AAV is selected from an AAV9, AAV1, AAV2, AAV4, AAV5, AAV6, AAV7, or AAV8, capsid serotype, a recombinant AAV (rAAV), or a functional variant of AAV9, AAV1, AAV2, AAV4, AAV5, AAV6, AAV7, or AAV8, including a self-complementary variant thereof.

4. The composition of any one of claims 1-3, wherein the human KCNN1 cDNA comprises a polynucleotide sequence encoding the polypeptide of SEQ ID NO: 1 or a functional variant or fragment having at least about 80%, or at least about 90%, or at least about 95%, or at least about 97%, or at least about 99% identity thereto.

5. The composition of any one of claims 1-4, wherein the human KCNN1 cDNA comprises a polynucleotide sequence of SEQ ID NO: 2 or a functional variant or fragment having at least about 80%, or at least about 90%, or at least about 95%, or at least about 97%, or at least about 99% identity thereto.

6. The composition of any one of claims 1-5, wherein the human KCNN1 cDNA comprises a codon optimized form of a polynucleotide sequence of SEQ ID NO: 2.

7. The composition of any one of claims 1-6, wherein the enhancer-promoter combination or promoter is selected from a cytomegalovirus (CMV) enhancer/promoter, a CMV enhancer fused to the chicken β-actin promoter (CAG), a chicken β-actin promoter (CBA), a simian vacuolating virus 40 (SV40) enhancer/promoter, a polyubiquitin C gene promoter (UBC), an elongation-factor 1α subunit (EF-1α) promoter, or a phosphoglycerate kinase promoter (PGK).

8. The composition of any one of claims 1-6, wherein the enhancer-promoter combination or promoter comprises a polynucleotide sequence of SEQ ID NO: 8 or a functional variant having at least about 80%, or at least about 90%, or at least about 95%, or at least about 97%, or at least about 99% identity thereto.

9. The composition of any one of claims 1-8, wherein the enhancer-promoter combination or promoter is suitable for use in humans.

10. The composition of any one of claims 1-9, wherein the enhancer-promoter combination or promoter is tissue-specific, inducible, or both tissue-specific and inducible.

11. The composition of claim 10, wherein the tissue-specific enhancer-promoter combination or promoter is a neuron-specific enhancer-promoter combination or promoter.

12. The composition of claim 11, wherein the neuron-specific enhancer-promoter or promoter is selected from a neuron-specific enolase (ENO2), a platelet-derived growth factor α-chain (PDGFA), a platelet-derived growth factor β-chain (PDGFB), a synapsin (SYN1), a methyl-CpG binding protein 2 (MECP2), a Ca2⁺/calmodulin-dependent protein kinase II (CAMK2G), metabotropic glutamate receptor 2 (GRM2), a neurofilament light (NEFL) or heavy (NEFH) chain, a proenkephalin (PENK), or an excitatory amino acid transporter 2 (SLC1A2).

13. The composition of any one of claims 1-12, wherein one or more of the ITRs comprises an ITR of the same serotype as the AAV capsid, comprises an ITR derived from the same serotype as the AAV capsid, comprises an ITR of a different serotype as the AAV capsid, or comprises an ITR derived from a different serotype as the AAV capsid.

14. The composition of any one of claims 1-13, wherein one or more of the ITRs comprises a polynucleotide sequence of SEQ ID NO: 5, SEQ ID NO: 6, or SEQ ID NO: 7 or a functional variant or fragment having at least about 80%, or at least about 90%, or at least about 95%, or at least about 97%, or at least about 99% identity thereto.

15. The composition of any one of claims 1-14, wherein the gene transfer construct further comprises one or more of an intron, a transcriptional termination signal, a polyadenylation (polyA) site, an miRNA, or a post-transcriptional regulatory element (PRE).

16. The composition of any one of claims 1-15, wherein the gene construct comprises, from 5′ to 3′: an ITR, a CMV enhancer/promoter, an intron, a human KCNN1 cDNA, a transcriptional termination sequence, a poly A site, and an ITR.

17. The composition of any one of claims 1-16, wherein the composition is suitable for intrathecal or intracerebroventricular delivery.

18. A pharmaceutical composition comprising the composition of any one of claims 1-17, and a pharmaceutically accepted excipient, carrier or diluent.

19. An ALS therapy comprising an effective amount of the pharmaceutical composition of claim 18.

20. A method for treating or preventing ALS in a subject in need thereof, comprising administering to the subject an effective amount of the pharmaceutical composition of claim 18 or the ALS therapy of claim 19.

21. A method for treating or preventing ALS in a subject in need thereof, comprising administering to the subject an effective amount of an ALS therapy, wherein the ALS therapy modulates the expression of KCNN1 and/or the activity of the gene product of KCNN1, or a fragment or variant thereof.

22. A method of treating or preventing ALS in a subject in need thereof comprising administering to the subject an effective amount of an ALS therapy, wherein the ALS therapy:
decreases and/or inhibits a neuron firing rate;
increases and/or stimulates a duration of afterhyperpolarization in a neuron;
decreases and/or inhibits membrane potential during the afterhyperpolarization phase in a neuron;
decreases and/or inhibits negative membrane potential further below resting potential during an afterhyperpolarization phase;
increases and/or prolongs an afterhyperpolarization phase;
increases and/or stimulates K+ efflux during afterhyperpolarization phase in a neuron;
increases and/or stimulates expression of KCNN1 gene or protein in a neuron;
increases and/or stimulates activity of small conductance calcium-gated K⁺ (SK) channels in a neuron;
increases and/or stimulates protection of motor neurons;
decreases translation of or increases and/or stimulates clearance of pathogenic protein; or
a combination thereof.

23. The method of claim 22, wherein the small conductance calcium-gated K⁺ (SK) channel is SK1 or a fragment or variant thereof.

24. The method of any one of claim 22 or 23, wherein the ALS therapy is:
a chemical compound that mimics a proteostatic protective effect of Kcnn1 overexpression;
a gene therapy;
a triazolo pyrimidine; or
one or more of N-{7-[1-(4-chloro-2-methylphenoxy)ethyl]-[1,2,4]triazolo[1,5-a]pyrimidin-2-yl}-N′-methoxy-formamidine [(-)CM-TPMF], chlorzoxazone, 5,6-Dichloro-1-ethyl-1,3-dihydro-2H-benzimidazol-2-one (DCEBIO), riluzole, and/or 1-ethyl-2benzimidazolinone (1-EBIO), or a pharmaceutically acceptable salt thereof.

25. The method of claim 24, wherein the gene therapy is a viral gene therapy.

26. The method of claim 25, wherein the viral gene therapy comprises a recombinant adeno-associated virus (rAAV).

27. The method of claim 26, wherein the rAAV is a human KCNN1-expressing rAAV or an rAAV expressing a fragment or variant of KCNN1.

28. The method of claim 25, wherein the viral gene therapy comprises a composition of any one of claims 1-17.

29. The method of claim 24, wherein the triazolo pyrimidine is selected from (-)CM-TPMF or a derivative of (-)CM-TPMF.

30. The method of any one of claims 20-29, further comprising:
administering an effective amount of at least one additional agent or
selecting a subject for administration of the ALS therapy, wherein the subject is undergoing treatment with at least one additional agent.

31. The method of claim 30, wherein the additional agent is a
benzothiazole (e.g. riluzole, or a derivative thereof);
antioxidant (e.g. edaravone, or a derivative thereof);
phenylbutyrate and/or taurursodiol:
antisense oligonucleotide, optionally suitable for intrathecal delivery;
tauroursodeoxycholic acid (TUDCA) and sodium phenylbutyrate (e.g. AMX0035, or a derivative thereof);
myeloperoxidase (MPO) enzyme inhibitor (e.g. verdiperstat, or a derivative thereof);
catalytically-active gold nanocrystal (e.g. CNMAU-8, or a derivative thereof);
sigma-1 receptor agonist (e.g. pridopidine, or a derivative thereof);
KCNQ activator (e.g. ezogabine, or a derivative thereof);
PIKfyve kinase inhibitor (e.g. YM201636, or a derivative thereof);
endosomal trafficking modulator;
electrokinetically altered aqueous fluid (e.g. RMS60, or a variant thereof);
anti-inflammatory agent;
cyclic nucleotide phosphodiesterase inhibitor (e.g. ibudilast, or a derivative thereof);
antisense oligonucleotide (e.g. directed to C9ORF72, e.g. WVE-004 or directed to SOD1 or FUS);
antibody (e.g. anti-CD40L, e.g. tegoprubart);
trehalose; or
combinations thereof.

32. The method of claim 30, wherein the additional agent is selected from N-{7-[1-(4-chloro-2-methylphenoxy)ethyl]-[1,2,4]triazolo[1,5-a]pyrimidin-2-yl}-N′-methoxy-formamidine [(-) CM-TPMF], chlorzoxazone, 5,6-Dichloro-1-ethyl-1,3-dihydro-2H-benzimidazol-2-one (DCEBIO), riluzole, and/or 1-ethyl-2benzimidazolinone (1-EBIO), or a pharmaceutically acceptable salt thereof.

33. The method of any one of claims 20-32, wherein the ALS therapy, the additional agent, or the ALS therapy and the additional agent is administered to the subject by at least one route selected from nasal, inhalational, topical, oral, buccal, rectal, pleural, peritoneal, vaginal, intramuscular, subcutaneous, transdermal, epidural, intratracheal, optic, intraocular, intracranial, intrathecal, intracerebroventricular, or intravenous.

34. The method of any one of claims 20-33, wherein the subject is a mammal.

35. The method of claim 34, wherein the mammal is a human.

36. The method of claim 35, wherein the human is about 40 to about 80 years old.

37. The method of any one of claims 20-36, wherein the ALS is sporadic ALS (sALS) or familial ALS (fALS).

38. The method of any one of claims 20-37, wherein the ALS is early, middle, or late stage.

39. The method of any one of claims 20-38, wherein the method prevents or retards progression from early to middle stage ALS or middle to late-stage ALS.

40. The method of any one of claims 37-39, wherein the subject demonstrates one or more of loss of strength; clumsiness; difficulty breathing; neurogenic atrophy; or loss of motor neurons in primary motor cortex, the brainstem, and/or the spinal cord.

41. The method of claim 40, wherein the subject presents one or more of the following genetic risk factors: C9orf72 hexanucleotide repeat expansion; one or more mutations in SOD1; one or more mutations in FUS; one or more mutations in TARDBP; or one or more mutations in other genes associated with or conferring risk for ALS.

42. The method of any one of claims 20-41, wherein the subject has failed and/or responded poorly to a previous therapy.

43. The method of any one of claims 20-42, wherein the subject has failed and/or responded poorly to a benzothiazole (e.g. riluzole) or an antioxidant (e.g. edaravone) or TUDCA/phenylbutyrate.

44. The method of any one of claims 20-43, wherein the subject demonstrates one or more of the following during or after treatment:
decreased neuron firing rate;
increased duration of afterhyperpolarization in a neuron;
decreased membrane potential during afterhyperpolarization phase in a neuron;
decreased negative membrane potential further below resting potential during the AHP phase;
increased and/or prolonged afterhyperpolarization phase;
increased and/or stimulated K+ efflux during afterhyperpolarization phase in a neuron;
increased and/or stimulated expression of KCNN1 in a neuron;
increased and/or stimulated activity of small conductance calcium-gated K⁺ (SK) channels in a neuron;
increased and/or stimulated protection of motor neurons;
increased and/or stimulated clearance of pathogenic protein.

45. The method of any one of claims 22-44, wherein the neuron is a motor neuron.

46. The method of any one of claims 20-45, wherein the method further comprises evaluating analysis of a biological fluid e.g., for biomarkers, e.g., one or more of NfL and GPNMB.

47. The method of claim 46, wherein the biological fluid is cerebrospinal fluid (CSF) or blood.

48. The method of any one of claims 20-45, wherein the method further comprises evaluating analysis of a brain image.

49. The method of claim 48, wherein the brain image is from one or more of computed tomography (CT), positron emission tomography (PET), or magnetic resonance imaging (MRI).

50. A method of making an ALS therapy, comprising:
(a) identifying the ALS therapy by:
(i) administering an effective amount of a test agent to a transgenic animal expressing G93A mutant human SOD1 or a wild-type animal;
(ii) identifying the test agent as an ALS therapy if the transgenic animal demonstrates one or more of increased expression of Kcnn1, increased amount or activity of the gene product of Kcnn1 or of a fragment or variant thereof, improved survival time, or decreased progression of paralysis, as compared to a control; and
(b) formulating the candidate agent for administration for the treatment of ALS.

51. A method of making an ALS therapy, comprising:
(a) identifying a candidate agent that modulates Kcnn1, comprising:
(i) providing a cell line expressing Kcnn1,
(ii) treating the cell line with one or more test agents,
(iii) measuring a voltage-independent potassium channel activity in the cell, and
(iv) identifying the test agent as a candidate agent, if voltage-independent potassium channel activity is increased in the presence of the test agent, compared to in the absence of the test agent and/or presence of a control agent that is known not to be a candidate agent; and
(b) administering an effective amount of the candidate agent to a transgenic animal expressing G93A mutant human SOD1 or a wild-type animal;
(c) classifying the candidate agent as an ALS therapy if the transgenic animal demonstrates one or more of increased expression of Kcnn1, increased activity of the gene product of Kcnn1, improved proteostatic clearance of the toxic protein(s), improved survival time to paralysis, or reduced progression of paralysis, as compared to a control; and
(d) formulating the ALS therapy for administration for the treatment of ALS.

52. A method of identifying an ALS therapy that is effective in treating or preventing ALS comprising administering an effective amount of the agent to an SOD1-mutant mouse and determining time to paralysis,
wherein an increased time to paralysis compared to an SOD1-mutant mouse that does not receive the agent indicates that the agent is an ALS therapy.

53. The method of any one of claims 50-52, wherein the ALS therapy is a gene therapy, a biologic agent, a small molecule, or a polynucleotide agent.

54. The method of claim 53, wherein the gene therapy is a viral gene therapy.

55. The method of claim 54, wherein the viral gene therapy is or comprises a recombinant adeno-associated virus (rAAV).

56. The method of claim 55, wherein the rAAV is a Kcnn1-expressing rAAV.

57. The method of claim 53, wherein the biologic agent is an antibody or peptide or small molecule.

58. The method of claim 53, wherein the polynucleotide agent is selected from mRNA, siRNA, shRNA, miRNA, or cDNA.

59. A pharmaceutical composition comprising a gene transfer construct, wherein the gene transfer construct comprises:
(a) a human KCNN1 cDNA, or functional variant thereof having a polynucleotide sequence encoding the polypeptide of SEQ ID NO: 1 or a functional variant having at least about 95%, identity thereto;
(b) an enhancer-promoter combination or a promoter operably linked to the human KCNN1 cDNA, or functional variant thereof, wherein the enhancer-promoter combination or promoter is capable of controlling expression of the human KCNN1 cDNA;
(c) an inverted terminal repeat (ITR) 5′ of the enhancer-promoter combination or promoter operably linked to the human KCNN1 cDNA, or functional variant thereof; and
(d) an inverted terminal repeat (ITR) 3′ of the human KCNN1 cDNA, or functional variant thereof;
wherein the gene transfer construct is an adeno-associated virus 9 (AAV9)

60. The pharmaceutical composition of claim 59, wherein the human KCNN1 cDNA comprises a polynucleotide sequence encoding the polypeptide of SEQ ID NO: 1.

61. The pharmaceutical composition of claim 59, wherein the human KCNN1 cDNA comprises a polynucleotide sequence of SEQ ID NO: 2 or a functional variant or fragment having at least about 95% identity thereto.

62. The pharmaceutical composition of claim 59, wherein the enhancer-promoter combination or promoter comprises a polynucleotide sequence of SEQ ID NO: 8 or a functional variant or fragment having at least about 95% identity thereto.

63. The pharmaceutical composition of claim 59, wherein the one or more of the ITRs comprises a polynucleotide sequence of SEQ ID NO: 5, SEQ ID NO: 6, or SEQ ID NO: 7 or a functional variant or fragment having at least about 95% identity thereto.

64. A method for treating or preventing ALS in a subject in need thereof, comprising administering to the subject an effective amount of the pharmaceutical composition or any one of claims 59-64.
